(19) United States
(12) Patent Application Publication
(10) Pub. No.: US 2025/0050036 A1
(43) Pub. Date: Feb. 13, 2025

(54) DRUG DELIVERY DEVICE WITH ADJUSTABLE INJECTION DEPTH

(71) Applicant: Sanofi, PARIS (FR)

(72) Inventors: Max Davis, Melbourn, Hertfordshire (GB); Thomas Mark Kemp, Melbourn, Hertfordshire (GB); Tom Lever, Melbourn, Hertfordshire (GB); Max Wehebrink, Melbourn, Hertfordshire (GB); Robbie Wilson, Melbourn, Hertfordshire (GB)

(21) Appl. No.: 18/719,688

(22) PCT Filed: Dec. 13, 2022
(86) PCT No.: PCT/EP2022/085643

§ 371 Date: Jun. 13, 2024

(30) Foreign Application Priority Data
Dec. 15, 2021 (EP) 21315274.7

(51) Int. Cl. A61M 5/46; A61M 5/32

(52) U.S. Cl. CPC A61M 5/46; A61M 5/3243

(57) ABSTRACT

A drug delivery device with an adjustable injection depth comprises a housing, a support element, and a container retaining space for receiving a container comprising a drug. The support element supports the container within the housing, and the drug delivery device comprises a spacer element. In a first state of the drug delivery device, a first axial position of the container relative to the housing is adjusted by the spacer element being in a first spacer position, and in a second state of the drug delivery device, a second axial position of the container is adjusted by the spacer element being in a second position within the housing or being outside of the housing. The first axial position enables a smaller injection depth of a needle coupled to the container compared to the injection depth enabled if the container is in the second axial position.

### CROSS REFERENCE TO RELATED APPLICATIONS

The present application is the national stage entry of International Patent Application No. PCT/EP2022/085643, filed Dec. 13, 2022, and claims priority to Application No. EP 21315274.7, filed Dec. 15, 2021, the disclosures of which are incorporated herein by reference.

### TECHNICAL FIELD

The disclosure relates to a drug delivery device with adjustable injection depth.

### BACKGROUND

A drug delivery device can be an autoinjector or a manually or semi-automatically operated device. An energy storing element is used in autoinjectors as well as in semi-automatically operated devices in order to deliver the driving force for the injection operation. The energy storing element may be biased in the factory or by the user prior to use. The drug may comprise insulin or GLP-1 (Glucagon-Like Peptide). However, other drugs may also be injected. Furthermore, other medical devices can include injectors, spraying devices, or inhalation devices.

It may be possible to adapt drug delivery devices to different target groups using needles of different length, i.e. a long needle for adults and a shorter needle for children. However, this may require to have detachable needles or to have different container types with integrated needles. Both options may be cumbersome and/or may have other disadvantages.

### SUMMARY

The disclosure describes a drug delivery device with adjustable injection depth. The device should be preferably easy and/or comfortable to use and/or comprise as few parts as possible. Furthermore, preferably easy adjustment of the injection depth should be possible. Furthermore, a corresponding method and/or corresponding items shall be provided, e.g., additional parts of the drug delivery device.

According to an embodiment, a drug delivery device is provided, comprising a housing. The housing may support inner components of the device. Alternatively or additionally, the housing may provide protection against environmental influences, e.g., mechanical influences, against humidity, etc.

According to an embodiment, the device may comprise a support element that is formed integral with the housing or that is mechanically connected to the housing. The support element may be adapted to the shape of at least a portion of the drug container and/or may be used to position the drug container relative to the housing.

According to an embodiment, the device may comprise a container retaining space for receiving a container comprising a drug or the container retaining space and the container comprising the drug. The container may be removable. Alternatively, the container may not be removable, e.g. as is the case in a disposable device.

According to an embodiment the support element may be configured to support the container within the housing. The support element may be the basis or a reference point for the container or for a datum of the container resulting in a reference injection depth. The reference injection depth may be adjusted, e.g. increased or decreased using e.g. spacer elements.

According to an embodiment, the drug delivery device may comprise at least one spacer element or may be adapted to interact with at least one spacer element. The spacer element(s) may have various shapes, e.g. a ramp shape, a distance element of constant thickness (disc-like, plate like), etc.

According to an embodiment, the drug delivery device may be configured such that in a first state of the drug delivery device, a first axial position of the container relative to the housing may be adjusted by the at least one spacer element being in a first spacer position. This embodiment is based on the consideration that positioning of the container results in positioning of a needle attached to the container or attachable to the container. Axial positioning of the needle may define the injection depth. Therefore, positioning of the container within the housing may be a simple way to adjust the injection depth. A first axial height of the spacer element may determine the axial position of the container in the first state. A second axial height of the spacer element or the missing spacer element may determine the axial position of the container in the second state.

According to an embodiment, in a second state of the drug delivery device, a second axial position of the container relative to the housing may be adjusted by the at least one spacer element being in a second position within the housing or being outside of the housing. There may be of course more than two states in order to adjust more than two injection depths. However, usage of only two states may simplify design and/or production and/or usage of the device. Thus, a height profile of the spacer element may be used to position the container axially and to adjust the injection depth thereby.

According to an embodiment, the first axial position may enable a smaller injection depth of a needle coupled to the container compared to the injection depth enabled if the container is in the second axial position. The smaller injection depth may be more appropriate for usage of the device for children, e.g. due to a thinner skin of children compared to the skin of adults.

According to an embodiment, the at least one spacer element may be configured to be translational movable crosswise, e.g. transverse or perpendicular, e.g. with an angle in the range of 80 degrees to 90 degrees, preferably about 90 degrees or 90 degrees, to a longitudinal axis of the drug delivery device from the first spacer position to the second spacer position or to a position outside of the drug delivery device.

According to an embodiment, at least one spacer element may be configured to be completely removable from the drug delivery device by a user. Thus, the spacer element may be an additional part of the device that is only delivered together with the device if needed. Thus, spacer elements are only produced for devices and users that need these spacer elements but not for users that do not need the spacer element since the “original” injection depth of the device is already appropriate. This may save material and/or production time and/or other efforts.

According to an embodiment, the at least one spacer element may be built into the housing. Thus, each device may comprise a spacer element independent of the question whether the user needs it or not. This approach may simplify logistics of the production and/or distribution of the device, e.g. by a reduced number of options.

According to an embodiment, the at least one spacer element may be configured to be movable from the first spacer position to the second spacer position using at least one operating element, preferably an operating element arranged on the outside of the housing. Thus, easy access and/or easy operation of the operating element may be possible.

According to an embodiment, the at least one spacer element may be configured to be translational movable from the first spacer position to the second spacer position or to a position outside of the drug delivery device. Thus, an easy design and/or a cheaper molding tool may be necessary since a translational movement may be realized by simpler means compared to other kinds of movements.

According to an embodiment, the spacer element may be a bifurcated spacer element comprising a basis portion, a first pronged portion and a second pronged portion extending in parallel from the basis portion and forming an intermediate space between the first pronged portion and the second pronged portion. Thus, the spacer element may have a simple structure and may therefore be easily to be produced.

According to an embodiment, the lateral width of the intermediate space may be greater than the lateral width or the diameter of a neck portion of the container at a position close to a larger diameter portion of a barrel of the container. Thus, the spacer element may be adapted to encompass the neck portion thereby moving a barrel portion proximally.
According to an embodiment, the spacer element may comprise at least one ramped portion on at least one of the following:
On a free end of the first pronged portion and/or on a free end of the second pronged portion, e.g. in order to ease insertion of the spacer, or
On at least one intermediate portion of the first pronged portion or on at least one intermediate portion of the second pronged portion, e.g. in order to ease switching between at least two spacer heights.

According to an embodiment, a second ramp may be arranged on at least one of the pronged portions between a first ramped portion, e.g. on the free end of the prong, and the basis portion.

According to an embodiment, the spacer element may comprise at least one portion of constant thickness on at least one of the following:
On the basis portion, e.g. forming an ergonomic gripping face, or
On the first pronged portion and/or on the second pronged portion, e.g. forming sections corresponding to a defined injection depth.

According to a further aspect, the at least one spacer element may be configured to be pivotable from the first position to the second position. This may allow solutions that need only little assembling space and/or solutions that ease switching, e.g. by reducing the switching force, between different insertion depths. A hinge, a pin, or other means may be used in order to provide a pivoting point.

According to an embodiment, the drug delivery device may comprise a rotatable operating feature configured to be rotated by a user of the drug delivery device. This may allow easy and ergonomic integration of the rotatable operating feature into a housing having e.g. an essentially cylindrical shape.

According to an embodiment, the rotatable operating feature may be configured to interact with the pivotable spacer element. Thus, the rotatable operating feature may be a ring comprising inner protrusion(s) adapted and/or configured to interact with the spacer element, thereby providing a simple mechanical interface.

According to an embodiment, the at least one spacer element may comprise a first class lever. The first class lever may comprise: an elongated operating portion, an elongated spacer portion configured to interact with at least one of the container or a carrier of the container, and a mounting portion arranged between the operating portion and the spacer portion and comprising a pivotable mounting element. Usage of a first class lever may allow to use little room necessary for the lever elements. Good force multiplying ratios may be reached using first class levers.

According to an embodiment, the at least one spacer element may comprise a second class lever. The second class lever may comprise: an elongated operating portion, an elongated spacer portion configured to interact with at least one of the container or a carrier of the container, and a mounting portion comprising a pivotable mounting element. The elongated spacer portion may be arranged between the elongated operating portion and the mounting portion. Good force multiplying ratios may be reached using second class levers.

According to a further embodiment, the at least one spacer element may comprise a third class lever, e.g. levers with the following sequence of points along the axis of the lever: fulcrum, input force and then load, e.g. syringe. Advanced solutions may be possible using third class levers.

Pairs of levers may be used. However, it is of course possible to use only one lever or to use more than two levers.

According to an embodiment, the at least one spacer element may comprise at least one cam element. The at least one cam element may comprise:
A mounting portion comprising a pivotable mounting element,
A curved outer surface adapted to interact with an operating element, and
An inner spacer portion configured to interact with at least one of the container and/or a carrier of the container.

The at least one cam element or cam spacer element may have a width, e.g. a radial width in the assembled state, that increases continuously up to a maximum width as the distance to the mounting portion increases. Cam elements may allow dedicated force profiles, e.g. in order to allow ergonomic adjustment of the injection depth.

The pivotable mounting element may be a pin configured to interact with a hole or a hole configured to interact with a pin. However, other simple solutions may also be used, e.g. a resilient element, e.g. a twistable element, a film hinge, etc.

The mounting portion of the cam spacer element may be arranged at one end of the cam spacer element, e.g. an end along the circumferential direction of the housing of the drug delivery device in an assembled state of the cam spacer element. However, other appropriate positions may also be used, e.g. arrangement of the mounting portion in an intermediate portion of the cam spacer element between the ends of the cam spacer element, e.g. ends along the circumferential direction of the housing of the drug delivery device in an assembled state of the cam spacer element.

Elongated parts of the spacers (lever(s), cam(s)) may allow larger force transmission ratios. Straight sections of the spacers (lever(s), cam(s)) may be easily to be manufactured, e.g. operation section(s). Curved sections of the spacer portion (lever(s), cam(s)) may be adapted to curvatures of container/barrel of container allowing larger areas for the application of the force, e.g. reducing the risk of breakage of the container (e.g. of a glass container).

According to an embodiment, the at least one spacer element may comprise a curved portion. According to an embodiment, the at least one spacer element may comprise at least one ramp feature configured to move or to allow to move the container or the container and a carrier of the container from the first axial position to the second axial position. There are synergistic effects if ramped portion are curved. The curvature of the curved portion may be adapted to the curvature of the outer diameter of container, e.g. the curvature of the at least one spacer element may be within a range of minus 10 percent to plus 10 percent of the curvature of the outer container surface, e.g. surface of a barrel or barrel portion and/or of a cylindrical portion, measured e.g. within a plane cross section perpendicular to the longitudinal axis of the drug delivery device. The same may apply to the radius of the container compared to the radius of the curved portion.

According to an embodiment, the at least one spacer element may comprise a proximal facing first face configured to abut the container and a distally facing second face configured to abut the housing or the support element or another element of the drug delivery device. The proximal facing first face of the spacer element (or spacer) may abut a distally facing face of the container, e.g. of a barrel of the container or of a flange of the container. Alternatively or additionally, the proximal facing first face of the spacer element may abut a distal element of a carrier for the container, e.g. distal arms or a distal rim of the carrier.

According to an embodiment, the container may comprise a barrel portion comprising a first diameter, a distal neck portion and a shoulder portion between the barrel portion and the distal neck portion. According to an embodiment, the neck portion may comprise a second diameter that is smaller than the first diameter. According to an embodiment, the first surface may be configured to abut the shoulder portion. In case of a glass container, the shoulder portion may be less prone to breakage than other portions. The container may e.g. be a syringe, e.g. comprising a proximal flange and/or an integrated needle, or a cartridge, e.g. comprising no proximal flange and/or comprising an adapter for an attachable and/or removable needle.

According to an embodiment, the container may comprise a barrel portion comprising a first diameter and a flange portion comprising a maximum second diameter that is greater than the first diameter. According to an embodiment, the first surface of the spacer element may be configured to abut the flange portion. There may be easier access to the flange than to the distal part of the container.

According to an embodiment, the drug delivery device may comprise a container carrier. The container carrier may comprise a main carrier portion encompassing the retaining space or the container, preferably a proximal flange portion and preferably at least one distal arm extending distally from the main carrier portion. According to an embodiment, the first surface of the spacer element may be configured to abut the distal end of the carrier, preferably a distal end of at least one of the at least one distal arms.

Alternatively, the second surface or face of the spacer element may be configured to abut to a proximal part of the container carrier, preferable a proximal part of the flange portion of the container carrier. This may provide synergistic effects if the first face of the spacer element abuts a flange of the container since the spacer is insertable between these two flanges or into an intermediate space between these two flanges. Support of the spacer element on the housing may be optionally in this embodiment.

According to an embodiment, the drug delivery device may comprise an axially movable needle protection element, e.g. a needle shroud. According to an embodiment, at least one resilient element, preferably only one resilient element, may be configured to bias the axially movable needle protection element in the distal direction. According to an embodiment, the drug delivery device may be configured such that a proximal end of the at least one resilient element is arranged on an abutting face that is located distally compared to the at least one spacer element or to a retaining space for the at least one spacer element. This may ensure that there is no detrimental interference between the resilient element and the spacer elements and/or operating element of the spacer elements.

According to an embodiment, the drug delivery device may comprise an axially movable needle protection element, e.g. a needle shroud. According to an embodiment, the drug delivery device may comprise at least two resilient elements configured to bias the axially movable needle protection element in the distal direction. According to an embodiment, the proximal ends of the at least two resilient elements may be arranged on at least one abutting face that is located proximally compared to the at least one spacer element or to a retaining space for the at least one spacer element. Alternatively or additionally, the at least two resilient elements may be arranged laterally with regard to the at least one spacer element or to a retaining space for the at least one spacer element and/or to an operating element of the at least one spacer element. This alternative technical solution may also ensure that there is no detrimental interference between the resilient element and the spacer elements and/or operating element of the spacer elements. Thus, there are at least two design options.

According to a further embodiment, the drug delivery device may comprise at least one holding element arranged within the housing and configured to bias the container or a carrier of the container in a distal direction. According to a further embodiment, the at least one holding element may comprises at least one or both of:
A resilient element allowing axial displacement of the container and/or of an optional carrier of the container, and/or
At least one rigid arm extending in the axial direction and carrying the resilient element.

The resilient element may comprise a flexible material, e.g. plastic or polymers. The resilient element may comprise a closed loop, a flexible arm, etc. Alternatively, compression springs or other spring elements may be used, e.g. springs comprising at least one, at least two or more than two windings.

According to a further embodiment, the holding element may comprise a proximal end cap of the drug delivery device, e.g. rear part of housing or casing. The rigid arms may extend distally from the proximal end cap.

According to a further embodiment, a bifurcated spacer element may be claimed separately. The bifurcated spacer element may comprise a basis portion, a first pronged portion and a second pronged portion extending in parallel from the basis portion and forming an intermediate space between the first pronged portion and the second pronged portion. Thus, the technical effects mentioned above for such a spacer element may also apply to the spacer element alone.

According to a further embodiment, the lateral width of the intermediate space may be greater than the lateral width or the diameter of a neck portion of the container at a position close to a larger diameter portion of the container, e.g. of a barrel of the container. The lateral width of the intermediate space may be e.g. in the range of 1 to 10 percent greater compared to the lateral width or the diameter of a neck portion of the container at the position mentioned above. Thus, again, the technical effects mentioned above for such a spacer element may also apply to the spacer element alone.

The spacer element may be used for a drug delivery device as mentioned above, especially as a translational movable spacer element. The technical effects mentioned above may also apply to the corresponding spacer element.

According to a further embodiment, a method for adjusting the injection depth of a drug delivery device may be claimed, especially of a drug delivery device according to any one of the claims and/or according to any one of the embodiments mentioned above. According to the further embodiment, at least one spacer element may be used to adjust the relative axial position of a container comprising a drug relative to a housing of the drug delivery device, especially relative to a support element that is formed integral with the housing or that is mechanically connected to the housing and that is configured to support the container within the housing. Thus, the technical effects mentioned above may also apply to the method.

The present application claims priority of EP 21315274.7, filed on Dec. 15, 2021, the disclosure of which is herewith explicitly incorporated by reference into the present disclosure for all legal purposes.

In the following, a set of aspects is disclosed. The aspects are numbered to facilitate referencing the features of one aspect in other aspects. The aspects form part of the disclosure of the present application and could be made subject to independent and/or dependent claims irrespective of what currently is claimed in the application and also independent of the references in brackets.

In a first aspect, a drug delivery device with adjustable injection depth comprises a housing, a support element integral with the housing or mechanically connected to the housing, and a container retaining space for receiving a container comprising a drug, wherein the support element is configured to support the container within the housing, wherein the drug delivery device comprises at least one spacer element or is adapted to interact with at least one spacer element,
wherein the drug delivery device is configured such that in a first state of the drug delivery device, a first axial position of the container relative to the housing is adjusted by the at least one spacer element being in a first spacer position, and that in a second state of the drug delivery device, a second axial position of the container relative to the housing is adjusted by the at least one spacer element being in a second position within the housing or being outside of the housing, wherein the first axial position enables a smaller injection depth of a needle coupled to the container compared to the injection depth enabled if the container is in the second axial position.

In a second aspect of the drug delivery device according to the first aspect, the at least one spacer element is configured to be completely removable from the drug delivery device by a user.

In a third aspect of the drug delivery device according to the first aspect or the second aspect, the at least one spacer element is built into the housing, and the at least one spacer element is configured to be movable from the first spacer position to the second spacer position using at least one operating element.

In a fourth aspect of the drug delivery device according to any one of the preceding aspects, the at least one spacer element is configured to be translational movable from the first spacer position to the second spacer position or to a position outside of the drug delivery device.

In a fifth aspect of the drug delivery device according to the fourth aspect, the spacer element is a bifurcated spacer element comprising a basis portion, a first pronged portion and a second pronged portion extending in parallel from the basis portion and forming an intermediate space between the first pronged portion and the second pronged portion, preferably the lateral width of the intermediate space is greater than the lateral width or the diameter of a neck portion of the container at a position close to a larger diameter portion of a barrel of the container.

In a sixth aspect of the drug delivery device according to the fifth aspect, the spacer element comprises at least one ramped portion on at least one of the following: on a free end of the first pronged portion or on a free end of the second pronged portion or on at least one intermediate portion of the first pronged portion or on at least one intermediate portion of the second pronged portion, and
wherein the spacer element comprises at least one portion of constant thickness on at least one of the following: on the basis portion or on the first pronged portion or on the second pronged portion.

In a seventh aspect of the drug delivery device according to any one of the first aspect to the third aspect, the at least one spacer element is configured to be pivotable from the first position to the second position.

In an eighth aspect of the drug delivery device according to the seventh aspect, the drug delivery device comprises a rotatable operating feature configured to be rotated by a user of the drug delivery device, and the rotatable operating feature is configured to interact with the pivotable spacer element.

In a ninth aspect of the drug delivery device according to the seventh aspect or the eighth aspect, the at least one spacer element comprises:
a) a first class lever comprising;
an elongated operating portion,
an elongated spacer portion configured to interact with at least one of the container or a carrier of the container, and
a mounting portion arranged between the operating portion and the spacer portion and comprising a pivotable mounting element,
b) a second class lever comprising:
an elongated operating portion,
an elongated spacer portion configured to interact with at least one of the container or a carrier of the container, and
a mounting portion comprising a pivotable mounting element,
wherein the elongated spacer portion is arranged between the elongated operating portion and the mounting portion,
c) at least one cam element comprising:
a mounting portion comprising a pivotable mounting element,
a curved outer surface adapted to interact with an operating element, and
an inner spacer portion configured to interact with at least one of the container or a carrier of the container.

In a tenth aspect of the drug delivery device according to any one of the seventh aspect to the ninth aspect, the at least one spacer element comprises a curved portion, and preferably the at least one spacer element comprises at least one ramp feature configured to move or to allow to move the container or the container and a carrier of the container from the first axial position to the second axial position.

In an eleventh aspect of the drug delivery device according to any one of the preceding aspects, the at least one spacer element comprises a proximal facing first face configured to abut the container and a distally facing second face configured to abut the housing or the support element or another element of the drug delivery device.

In a twelfth aspect of the drug delivery device according to the eleventh aspect, the container comprises barrel portion comprising a first diameter, a distal neck portion and a shoulder portion arranged between the barrel portion and the distal neck portion, the neck portion comprises a second diameter that is smaller than the first diameter, and the first surface is configured to abut the shoulder portion.

In a thirteenth aspect of the drug delivery device according to the eleventh aspect, the container comprises a barrel portion comprising a first diameter and a flange portion comprising a maximum second diameter that is greater than the first diameter, and the first surface is configured to abut the flange portion.

In a fourteenth aspect of the drug delivery device according to any one of the eleventh aspect to the thirteenth aspect, the drug delivery device comprises a container carrier comprising a main carrier portion encompassing the retaining space or the container, preferably a proximal flange portion and preferably at least one distal arm extending distally from the main carrier portion, the first face is configured to abut the distal end of the container carrier, preferably a distal end of at least one of the at least one distal arms,
or the second face is configured to abut to a proximal part of the container carrier, preferably a proximal part of the flange portion of the container carrier.

In a fifteenth aspect of the drug delivery device according to any one of the preceding aspects, the drug delivery device comprises an axially movable needle protection element, and
a) at least one resilient element, preferably only one resilient element, configured to bias the axially movable needle protection element in the distal direction,
wherein the drug delivery device is configured such that a proximal end of the at least one resilient element is arranged on an abutting face that is located distally compared to the at least one spacer element or to a retaining space for the at least one spacer element, or
b) wherein the drug delivery device comprises at least two resilient elements configured to bias the axially movable needle protection element in the distal direction,
wherein the proximal ends of the at least two resilient elements are arranged on at least one abutting face that is located proximal compared to the at least one spacer element or to a retaining space for the at least one spacer element, and
wherein the at least two resilient elements are arranged laterally with regard to the at least one spacer element or to a retaining space for the at least one spacer element and/or to an operating element of the at least one spacer element.

The making and using of the presently preferred embodiments are discussed in detail below. It should be appreciated, however, that the present disclosure provides many applicable concepts that can be embodied in a wide variety of specific contexts. The specific embodiments discussed are merely illustrative of specific ways to make and use the disclosed concepts, and do not limit the scope of the claims or aspects.

Moreover, same reference numerals refer to same technical features if not stated otherwise. As far as “may” is used in this application it means the possibility of doing so as well as the actual technical implementation. The present concepts of the present disclosure will be described with respect to preferred embodiments below in a more specific context namely drug delivery devices, especially drug delivery devices for humans or animals. The disclosed concepts may also be applied, however, to other situations and/or arrangements as well, e.g. for lancets, other injectors, spraying devices or inhalation devices.

The foregoing has outlined rather broadly the features and technical advantages of embodiments of the present disclosure. Additional features and advantages of embodiments of the present disclosure will be described hereinafter, e.g. of the subject-matter of dependent claims. It should be appreciated by those skilled in the art that the conception and specific embodiments disclosed may be readily utilized as a basis for modifying or designing other structures or processes for realizing concepts which have the same or similar purposes as the concepts specifically discussed herein. It should also be recognized by those skilled in the art that equivalent constructions do not depart from the spirit and scope of the disclosure, such as defined in the appended claims.

### BRIEF DESCRIPTION OF THE FIGURES

For a more complete understanding of the presently disclosed concepts and the advantages thereof, reference is now made to the following description in conjunction with the accompanying drawings. The drawings are not drawn to scale. In the drawings the following is shown in:

FIG. 1 a drug delivery device,

FIG. 2A a drug delivery device according to a second embodiment comprising a spacer unit in a deactivated state shortly before reaching of the injection depth and shortly before activation of the driving mechanism,

FIG. 2B the drug delivery device according to FIG. 2A at reaching the injection depth and at the beginning of the injection of the drug,

FIG. 3A the drug delivery device according to the second embodiment with the spacer unit in an activated state shortly before reaching of the injection depth and shortly before activation of the driving mechanism,

FIG. 3B the drug delivery device according to FIG. 3A at reaching the injection depth and at the beginning of injection of the drug,

FIG. 4A a transversal cross section through a drug delivery device according to a third embodiment adapted for insertion of a bifurcated spacer but with the bifurcated spacer outside of the device,

FIG. 4B a longitudinal cross section of the drug delivery device according to FIG. 4A,

FIG. 4C a transversal cross section through the drug delivery device according to the third embodiment with the bifurcated spacer laterally inserted into the device,

FIG. 4D a longitudinal cross section of the drug delivery device according to the FIG. 4C,

FIG. 5A a longitudinal cross section of a drug delivery device according to a fourth embodiment,

FIG. 5B a longitudinal cross section of a drug delivery device according to a fifth embodiment,

FIG. 6A a perspective view of a drug delivery device according to a sixth embodiment configured to receive a bifurcated spacer element comprising two different spacer thicknesses,

FIG. 6B a longitudinal section of the drug delivery device according to FIG. 6A,

FIG. 7A a perspective view of a drug delivery device according to a seventh embodiment comprising two pivotable spacer arms (first class lever),

FIG. 7B a perspective top view of the drug delivery device according to FIG. 7A,

FIG. 8A a perspective view of the device of the seventh embodiment with the spacer arms in an inactivated first position,

FIG. 8B a side view of the device of the seventh embodiment with the spacer arms in the inactivated first position,

FIG. 8C a bottom view of the device of the seventh embodiment with the spacer arms in the inactivated first position,

FIG. 9A a perspective view of the device of the seventh embodiment with the spacer arms in an activated second position,

FIG. 9B a side view of the device of the seventh embodiment with the spacer arms in the activated second position,

FIG. 9C a bottom view of the device of the seventh embodiment with the spacer arms in the activated second position,

FIG. 10A a bottom view of a drug delivery device according to an eighth embodiment comprising two pivotable spacer arms (second class lever) in an inactivated first position,

FIG. 10B a bottom view of the device according to FIG. 10A with the spacer arms in an activated second position,

FIG. 11A a bottom view of a drug delivery device according to a ninth embodiment comprising two cam spacer elements being in an inactivated first position,

FIG. 11B a bottom view of the device according to FIG. 11A with the cam spacer elements in an activated second position,

FIG. 11C a bottom view of a drug delivery device according to a tenth embodiment comprising more than two cam spacer elements, and

FIG. 12 a perspective view of a drug delivery device comprising a ring feature.

### DETAILED DESCRIPTION

Reference may be made to a cylindrical coordinate system, i.e. each position may be defined by three coordinates: axial value (height, distance to zero plane), radial distance to axis and angle between current radial position and a plane that is defined as having angle zero. In this document the words “in an axial position” may mean having an axial coordinate.

The distal end D may be an end that is closer to a needle compared to a proximal end P.

Certain embodiments in this document are illustrated with respect to an injection device comprising a movable axial needle shroud used as an activation element, e.g. an autoinjector. Reference is made to WO 2015/004052 A1 in this regard which is included by reference for all legal purposes.

However, other embodiments may relate to drug delivery devices comprising other activation mechanism or operated by a manual driving force. Reference is made e.g. to WO 2014/033195 A1 or to WO 2014/033197 A1 in this regard which are included by reference for all legal purposes. The injection button may provide at least one user interface member for initiating and/or performing a dose delivery operation of the drug delivery device. The (dial) grip or knob may provide a user interface member for initiating and/or performing a dose setting operation using a dose setting surface, e.g. the circumferential surface of the (dial) grip or knob. A delivery surface may be used to initiate dose delivery. The delivery surface may be the proximal P surface of the (dial) grip or knob.

The device may be of the dial extension type, i.e. its length may increase during dose setting or dose dialing. Other injection devices with the same kinematical characteristic of the dial extension and button during dose setting and dose expelling operational mode are known as, for example, Kwikpen® and Savvio® device marketed by Eli Lilly as well as FlexPen® FlexTouch® and Novopen® 4 device marketed by Novo Nordisk or devices of other manufacturers. An application of the general principles disclosed herein to these devices therefore appears straightforward and further explanations will be omitted. Alternatively, the proposed concepts may be used in devices that are not of the dial extension type but include for instance torsion spring that are biased by rotation of a dial knob. Moreover, fully mechanically driven or electromechanically driven drug delivery devices may be used, e.g. comprising an electrical motor. A distance sleeve may be used in order to have a reference injection depth that may be adjusted using the spacer units, e.g. in case that other actuating elements than a movable needle shroud (needle protection element) are used.

However, the general principles of the present disclosure are not limited to that kinematical characteristic. Certain other embodiments may be conceived, e.g. for application to other injection devices of Sanofi or devices of other manufacturers where there is a container for a drug that may be positioned axially relative to a housing of the drug delivery device in order to adjust an injection depth.

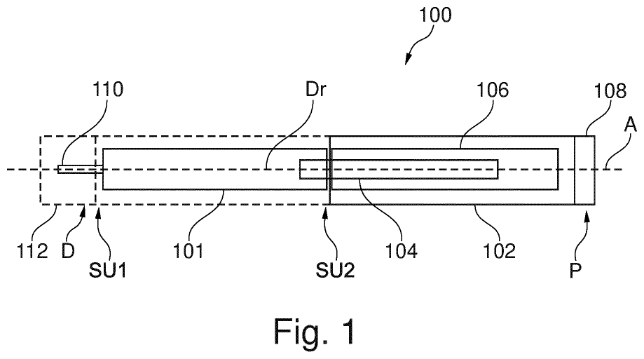

FIG. 1 illustrates a first embodiment of a drug delivery device 100 that may comprise a container retaining member 101 retaining a drug container (e.g. a syringe or a cartridge) and a main housing part 102. The container may comprise a drug Dr. Main housing part 102 may house or surround the container retaining member 101 and/or the container completely or partially and may comprise further parts of the drug delivery device 100. Alternatively, the main housing part 102 may be connected to the container retaining member 101 but may not surround it and even may not surround a part of the container retaining member 101, see dashed line in FIG. 1.

A longitudinal axis A of drug delivery device 100 is illustrated in FIG. 1 by a dashed straight line.

Within the main housing part 102 the following components may be arranged:
A piston rod 104 that is adapted to move a piston that may be arranged within container retaining member 101,
A driving mechanism 106 for the piston rod 104. The driving mechanism 106 may comprise an energy storing element, for instance a spring that is loaded manually before each use. Alternatively, the energy storing element may be loaded for instance during assembling of drug delivery device 100. Alternatively, a manually driven driving mechanism may be used, e.g. without an energy storing element that is used to drive piston rod 104.
For instance at a proximal end P, an actuating element 108 that is used for the initiation of a movement of piston rod 104 into the container retaining member 101, whereby the driving mechanism 106 is used. Alternatively, an autoinjector device may be used that is actuated by an axial movement of a movable needle shroud (not shown). Actuating element 108 may be used to dial the size of a dose of drug Dr in some embodiments.
A cap 112 that may be attached to main housing part 102 or to another part of drug delivery device 100. Cap 112 may be an outer cap that may include a smaller inner cap which protects a needle 110 directly.

If drug delivery device 100 is not an autoinjector, a dial sleeve may be screwed out of main housing 102 and may be pressed by a user in order to move plunger 104 distally and to inject drug Dr. Drug delivery device 100 may be a single use or a multiple use device.

Drug Dr may be dispensed from the container through a needle 110 or through a nozzle that is connectable and/or connected to the distal end D of drug delivery device 100. Needle 110 may be changed before each use or may be used several times.

Drug delivery device 100 may comprise an electronic unit that is mechanically connected to a proximal end region P of drug delivery device 100, for instance to a proximal end region P of actuating element 108. The electronic unit 120 may be used not only for drug delivery device 100 but also for other drug delivery devices that are similar or identical to drug delivery device 100. Alternatively, the electronic unit may be an integrated part of the drug delivery device 100 The electronic unit may be used to monitor drug delivery, e.g. amount of dose, time and date.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codeable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des (B30) human insulin, Lys (B29) (N-tetradecanoyl)-des (B30) human insulin (insulin detemir, Levemir®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N—(N-lithocholyl-gamma-glutamyl)-des (B30) human insulin; B29-N-(ω-carboxyheptadecanoyl)-des (B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

One of the basic concepts proposed in this application is to use spacer units or distance elements in order to position a syringe or another container comprising a drug Dr, e.g. a cartridge within the housing or relative to the housing 102. There may be several appropriate positions for placing the spacer unit, e.g. at a distal part of the syringe/container, see spacer unit SU1, at a proximal part of the syringe/container, see spacer unit SU2, e.g. at a flange of a syringe. However, other positions of the spacer unit may also be appropriate in order to position the container, e.g. a syringe, relative to housing 102. Thus, a container carrier may carry the container. The container carrier may allow positioning of the spacer units between a distal part of the container and a proximal part of the container, e.g. in the middle portion of the container.

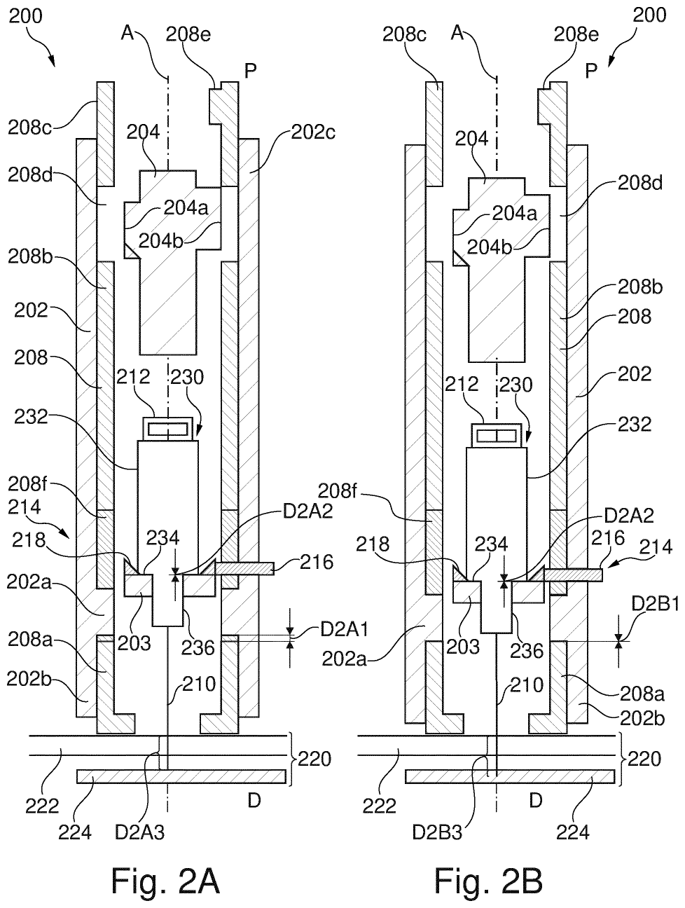

Detailed embodiments of spacer units SU1 to SU2 are described in the following FIGS. 2A to 12. Other embodiments are of course also possible.

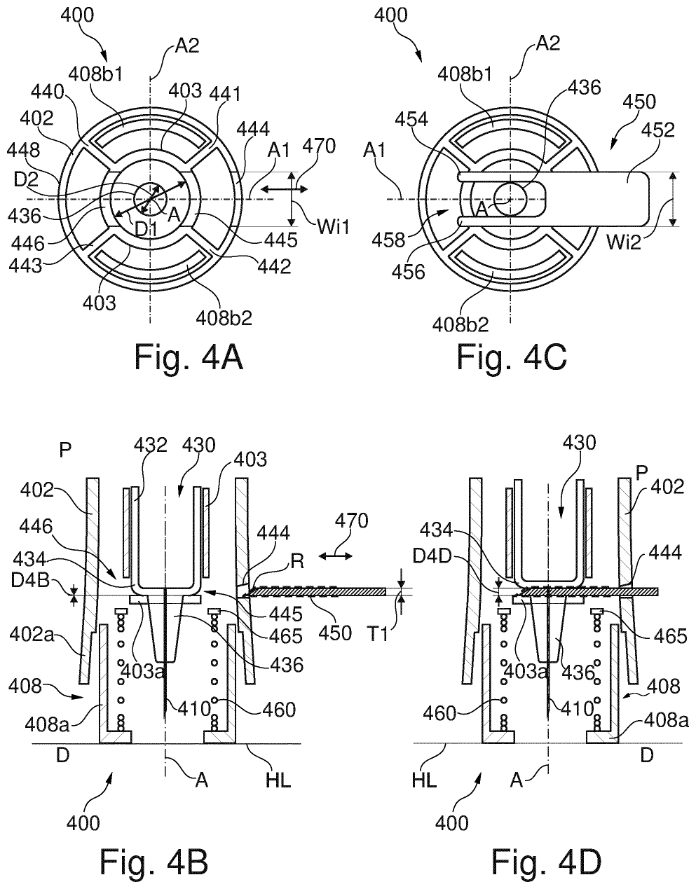

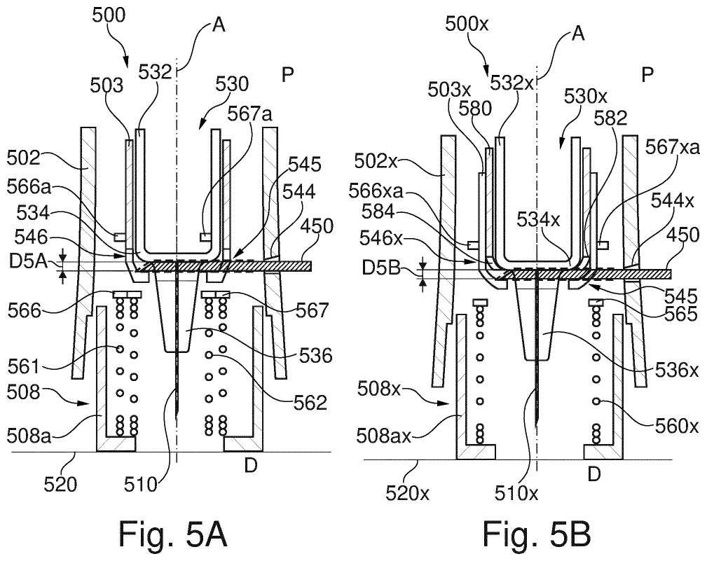

FIG. 2A illustrates a drug delivery device 200 according to a second embodiment comprising a spacer unit 214 in a deactivated state shortly before reaching of the injection depth and shortly before activation of the driving mechanism. Drug delivery device 200, e.g. drug delivery device 100, may comprise:
A main housing part 202, e.g. a cylindrical housing part extending from a proximal end P to a distal end,
A central part 203 of main housing part 202, e.g. a further cylindrical part or a disc like part that may be hold by axial/radial ribs and/or a flat part arranged perpendicular with respect to axis A, preferably coaxially to main housing part 202, see e.g. FIGS. 4A to 4D,
A piston rod 204, e.g. comprising a hollow or solid cylindrical main part and configured to interact with a piston (not shown) within the drug container in order to move the piston distally thereby expelling the drug (out-dosing),
A driving mechanism (not shown) for driving piston rod 204 distally,
A needle shroud 208 for protecting a needle, e.g. for preventing needle sticks,
A drug container, e.g. a syringe 230, and
An optional resilient element 212 arranged within the housing and configured to bias the container (e.g. syringe 230) or a carrier (see e.g. FIG. 5B, 580) of the container in a distal direction.

Main housing part 202 may comprise:
A distal portion 202b encompassing a distal portion 208a of needle shroud 208 and/or providing attachment means for a distal cap (not shown) of drug delivery device 200,
A proximal portion 202c, e.g. a portion that closes a proximal end of device 200. Alternatively, a rear housing part may be used to close the housing of drug delivery device 200 proximally.
A main portion arranged between distal portion 202b and proximal portion 202c.

The resilient element 212 may be arranged on the rear housing part or directly on housing part 202. A rear housing part may comprise a proximal closure portion and at least one arm, e.g. a rigid arm, or at least two arms, e.g. rigid arms, extending from the closure portion distally and preferably parallel to longitudinal axis A of drug delivery device 200. The at least one arm may be a rigid arm carrying a distal resilient element 212, e.g. at least one flexible loop, at least one flexible arm, etc. Alternatively, the at least one arm may be a flexible arm forming the resilient element that biases the container and/or the carrier of the container in the distal direction.

A protrusion 202a may be arranged on the main housing part 202 between distal portion 202b and the main portion of housing part 202. Protrusion 202a may be directed radially inwards. Protrusion 202a may interact with a distal aperture 208f that is described in more detail below and that may be arranged within needle shroud 208 near the distal portion 208a of needle shroud 208.

Piston rod 204 may comprise a first protrusion 204a extending radially outwards in a first direction and a second protrusion 204b extending radially outwards in a second direction that is opposite to the first direction. However, other design of the proximal part of piston rod 204 may also be used.

The driving mechanism (not shown) may comprise a resilient element, e.g. a compression spring, a tension spring or a torsion spring. Alternatively or additionally, the driving mechanism may allow manual driving of piston rod 204. The driving mechanism may be activated using protrusions 204a, 204b, 208e as well as aperture 208d. Alternatively, driving mechanism may be actuated according to other appropriate activation mechanisms and/or electronically.

Needle shroud 208 may comprise:
A distal portion 208a comprising an aperture through which needle 210 may reach a skin 220 of a user, e.g. of a patient, of drug delivery device 200,
At least one arm 208b or at least two arms 208b (e.g. a pair of arms) extending proximally from distal portion 208a and parallel to longitudinal axis A, and
At least one proximal portion 208c.

Proximal portion 208c may comprise:
A proximal aperture 208d, and
A protrusion 208e.

Distal portion(s) 208a may comprise the at least one distal aperture 208f. As already mentioned above, distal portion 208a may cooperate with protrusion 202a in order to limit axial movement of needle shroud 208 relative to main housing part 202 in the distal D direction and/or in the proximal P direction.

Needle 210 may be an integral part of the drug container, e.g. in case of a syringe 230. Alternatively, needle 210 may be attachable to and detachable from the drug container, e.g. by a screw connection, a Luer-Lock (Luer taper) or another appropriate detachable fastening element. Needle 210 may have a diameter in the range of 25 Gauge (outer diameter 0.5 millimeter) to 30 Gauge (outer diameter 0.3 millimeter) or another appropriate range. The overall length of needle 210 may be in the range of 10 mm to 30 mm or of 7 mm to 20 mm, e.g. 12.7 mm. The maximum injection depth of a drug delivery device, e.g. using no spacer element or using inactivated spacer element may be in the range of 3 mm to 15 mm.

The design of the device may be such that the device activates at a nominal depth of 5 mm+/−2 mm, and may reach full insertion at 7 mm+/−1 mm. This may be an effective range of 3 mm to 8 mm.

For thin skin, e.g. in pediatric applications, it may be considered to reduce the range via a shim or another distance element to 2 mm to 7 mm, that is, shifting the nominal activation depth to 4 mm+/−2 mm, and the full insertion depth to 6 mm+/−1 mm.

The maximum injection depth and/or insertion depth may be reduced by the spacer element by a length within the range of 1 mm to 3 mm. Injection into muscles may be detrimental. Therefore short maximum injection depths, e.g. less than 6 mm may be used without folding the skin or pressing the skin during injection.

Alternatively, longer maximum injection depths may be used when the skin is folded/pressed during injection, e.g. injection depths of more than 6 mm, more than 7 mm, more than 8 mm, more than 9 mm or more than 10 mm. Nominal depths of larger than the current 7 mm+/−2 mm at full depth may be permissible e.g. in people with high subcutaneous (SC) layer, although this may be mainly a clinical issue. Again, these maximum injection depths may be reduced by the spacer element by a length within the range of 1 mm to 3 mm to mention only one practical range. Other ranges may be used as well.

A resilient syringe holder 212, e.g. the resilient element mentioned already above, may be used to bias the container, e.g. syringe 230 distally. Thus, drug container, e.g. syringe 230 may have an appropriate “play” in the proximal direction allowing spacer element(s) to displace the drug container, e.g. in the proximal direction P and/or in the distal direction D thereby adjusting the injection depth as will be described in the following in more detail.

Spacer (distance) unit 214 may be arranged on a distal end of the drug container, e.g. syringe 230. Other appropriate positions are possible as well as mentioned above. In the present second embodiment, spacer unit 214 comprises:
An optional operating element 216, and
At least one spacer (distance) portion 218 (e.g. ramped portion or a flat portion)

Skin 220 may be the skin of child, e.g. of a person of less than 14 years, of less than 10 years, or even of less than 5 years. Alternatively, skin 220 may be the skin of a baby, e.g. having an age of less than 1 year. The proposed concepts may allow to adjust the injection depth depending on the age of the patient. If no adjustment is made, drug delivery device 200 may be used for adults and for teenagers. Skin 220 may comprise:
An upper layer 222 comprising the free surface of skin 220 and adjacent regions, and
A target layer 224 that is below upper layer 222, and/or
An optional intermediate layer that is arranged between upper layer 222 and target layer 224.

A typical depth of target layer 224 may be in the range of 5 mm to 8 mm for adults and in the range of 2 to 5 mm for children.

The design of the device may be chosen such that the device activates at a nominal depth of 5 mm+/−2 mm, and reaches full insertion depth at 7 mm+/−1 mm. This is an effective range of 3 mm to 8 mm.

The depth of the subcutaneous (SC) layer thickness may be a clinical issue. The minimum bound of target layer 224 may be 2 mm or may be more than 2 mm.

Syringe 230 may comprise a larger diameter barrel portion 232, a shoulder 234, and a smaller diameter distal portion 236 (neck, cone). The smaller diameter portion 236 may have a smaller diameter compared to the diameter of the barrel portion 232, e.g. less than two thirds or less than half of the value of the larger diameter.

There may be a first remaining distance D2A1 between a distal edge of aperture 208f and a distal edge of protrusion 202a. Distance D2A1 may be less than 0.5 mm. Furthermore, in the inactivated state of spacer unit 214, a first axial distance D2A2 between shoulder 234 of syringe 230 and a proximal facing face of central part 203 may be 0 (zero) mm. A resulting insertion depth D2A3 may be less than 0.5 mm compared to the maximum insertion depth (injection depth) in the inactivated state of spacer unit 214.

FIG. 2B illustrates the drug delivery device 200 according to FIG. 2A at reaching the injection depth D2B3 and at the beginning of the injection of drug Dr. Activation may result from the relative positions of protrusion 208e and/or aperture 208d relative to protrusion 204a and/or 204b. A second remaining distance D2B1 is now e.g. 0 mm, i.e. distal edge of aperture 208f may abut to distal edge of protrusion 202a. Distance D2A2 remains the same as in the state illustrated in FIG. 2A, e.g. 0 mm for maximum insertion depth, since spacer unit 214 is still in its inactivated state. Consequently, insertion depth D2B3 is reached before and during injection, i.e. the injection depth. Injection depth D2B3 may be greater than insertion depth D2A3, e.g. by 0.5 mm in the embodiment. Resilient element 212 has a resiliency that is lower than the resiliency of the skin. Thus, syringe 230 is not replaced proximally relative to housing 202 during injection.

In an alternative embodiment, a spacer element may be introduced only from one side, e.g. from the left side. The spacer element may be inserted e.g. not reaching axis A or e.g. only up to axis A. Alternatively, the spacer element may be inserted further, e.g. crossing axis A and/or via the opposition side face of container/syringe 230 compared to the side face from which the spacer element is inserted. This may be true for “external” spacer elements (e.g. completely removable from the device 200) as well as for built-in spacer elements.

The spacer element may be a spacer element that is moved translational. This spacer element may be part of the drug delivery device, e.g. assembled into drug delivery device 200, or it may be a separate part forming a set together with drug delivery device 200 wherein the spacer element may be removed from drug delivery device 200 if no spacer element is needed, e.g. without using tools and/or without destroying the spacer element and/or drug delivery device 200.

Alternatively, the spacer element be a spacer element that is pivoted and/or rotated. Again, this pivoted or rotated spacer element may be part of drug delivery device 200, e.g. assembled into drug delivery device 200.

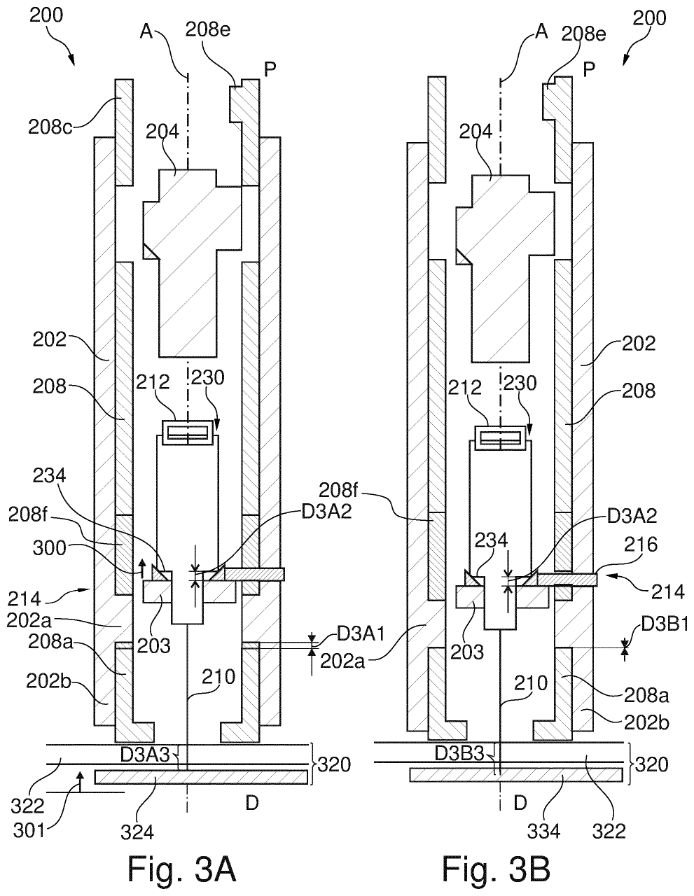

FIG. 3A illustrates drug delivery device 200 with spacer unit 214 in an activated state shortly before reaching of the injection depth and shortly before activation of the driving mechanism. Activation of spacer unit 214 moves distance portion(s) 218 between central part 203 and shoulder 234 of syringe 230 thereby shifting syringe 230 axially, e.g. proximally, by an axial shift 300. Axial, e.g. proximal, shift 300 of syringe 230 may result in an axial shift 301 of needle 210 thereby reducing the injection depth by an amount that corresponds or is equal to the value of axial shift 300, 301. A first remaining distance D3A1, i.e. between distal edge of aperture 208f and distal edge of protrusion 202a, is again e.g. equal to D2A1 as mentioned above, e.g. 0.5 mm. A second axial distance D3A2 between shoulder 234 of syringe 330 and proximal facing face of central part 303 equals to the value of axial shift 300, 301. Therefore, an insertion depth D3A3 that is the injection depth is less than the insertion depth D2A3 because of the activated state of spacer unit 214.

FIG. 3B illustrates drug delivery device 200 according to FIG. 3A at reaching the injection depth and at the beginning of injection of drug Dr. Again, activation may result from the relative positions of protrusion 208e and/or aperture 208d relative to protrusion 204a and/or 204b. A second remaining distance D3B1, i.e. between distal edge of aperture 208f and distal edge of protrusion 202a, may be similar to distance D2B1, e.g. 0 mm since the distal edge of aperture 208f abuts to distal edge of protrusion 202a. A distance D3A2 remains the same, e.g. corresponding to axial shift 300, 301 since spacer unit 214 is still in the activated state. Thus, insertion depth D3B3 during injection, e.g. the injection depth is less than injection depth D2B3 because of activated state of spacer unit 214. Thus, the activated state of spacer unit 214 may make drug delivery device 200 appropriate for injections of drugs Dr into the skin of children.

FIG. 4A illustrates a transversal cross section through a drug delivery device 400 according to a third embodiment adapted for insertion of a bifurcated spacer 450 but with the bifurcated spacer 450 outside of device 400.

Drug delivery device 400 is illustrated in more detail compared to drug delivery device 100 or drug delivery device 200. However, the concept described in the description of FIGS. 4A to 4D may be also applied in drug delivery device 100 or 200. Drug delivery device 400 comprises:
A main housing part 402 that may be similar to housing part 102, 202, etc.
A central part 403 of main housing part, e.g. a cylindrical part arranged coaxially to housing part 402,
A needle shroud 408, e.g. corresponding e.g. to needle shroud 208, and
A drug container, e.g. a syringe 430.

Furthermore, drug delivery device 400 may comprise all or some of the parts mentioned above for drug delivery device 100 or 200, e.g. a piston rod, a drive mechanism, etc.

Central part 403 may comprise a cylindrical main portion that encompasses only a distal portion of container/syringe 230 or that encompasses also a main portion of container/syringe 230. Central part 403 may be hold by axial ribs, see e.g. FIG. 4A, e.g. by at least two, at least three or at least four axially and radially extending ribs 440 to 444, in the following axial/radial ribs 440 to 444. A bottom 403a of central part 403 may be connected with central part 403, e.g. integrally formed with central part 403. However, the connection may be outside of the plane that is illustrated in FIGS. 4B and 4C.

Needle shroud 408 may comprise a distal portion 408a and arms 408b1, 408b2, e.g. a pair of arms. Distal portion 408a is pressed against the skin of a patient during injection, see horizontal lines HL in FIGS. 4B and 4D.

Syringe 430 may comprise a barrel 432, a shoulder 434 and a distal portion 436 (neck, cone). Distal portion 436 may comprise a smaller outer diameter D2 on its proximal end compared to the outer diameter D1 of barrel 432. Diameter D2 may be less than two thirds of diameter D1 or less than half of diameter D1 thereby providing enough room to push barrel 432 proximally using prongs 454, 456 of a bifurcated spacer 250.

Central portion 403 may be hold by at least two, at least three or at least four ribs 440 to 443 extending axially and radially. Axial and radial ribs 440 to 444 may be spaced equidistantly in circumferential direction, e.g. at angles of 45, 135, 215 and 305 degrees relative to a first transversal axis A1 and counted in counter clockwise direction. A second transversal axis A2 is arranged perpendicular (90 degrees) with respect to first transversal axis A1. Both axis A1 and A2 include angles of 90 degree with longitudinal axis A. In the embodiment, arm 408b1 of needle shroud 408 is arranged between ribs 440 and 441. Arm 408b2 of needle shroud 408 is arranged between ribs 442 and 443.

A first aperture 444 is arranged within the right wall of main housing part 402 extending circumferentially, e.g. forming a transversal slit with respect to longitudinal axis A. A second aperture 445 is arranged within the right wall of central part 403 at the same axial position as aperture 444. A third aperture 446 is arranged within the left wall of central part 403 at the same axial position as aperture 444.

First aperture 444, second aperture 445 and third aperture 446 are each centered (symmetrically arranged) with regard to axis A1. Each aperture 444, 445 and 456 has the same lateral width Wi1 that is slightly greater than a width Wi2 of spacer 450 as mentioned below and as illustrated in FIG. 4C.

Apertures 444 to 446 are aligned along first transversal axis A1 enabling insertion of spacer 450 as illustrated in FIGS. 4C and 4D. There is no fourth aperture in the region of a wall portion 448 that is arranged on the first transversal axis A1 opposite to apertures 444, 445 and 446. Thus, wall portion 448 may form a stop surface for insertion of bifurcated spacer element 450. Other stopping means are possible as well. However, in another embodiment a fourth aperture may be arranged in wall portion 448 similar to aperture 444 enabling e.g. introduction of spacer 450 from two different sides or advanced support of spacer 450 within housing 402.

Thus, apertures 444, 445 are arranged between ribs 441 and 442. Aperture 446 is arranged between ribs 440 and 443. Therefore, arms 408b1 and 408b2 do not interfere with room used for the insertion and/or the removal and/or translation of spacer 450 into apertures 444 to 446, see directions 470.

FIG. 4B illustrates a longitudinal cross section of drug delivery device 400 according to FIG. 4A. Bifurcated spacer 450 is illustrated on the right side of main housing part 402. Ramps R on the distal ends (free ends) of prongs 454 and 456 may ease insertion of spacer 450 between shoulder 434 and proximal face of bottom 403a of central part 403. Spacer 450 may have a constant thickness T1, e.g. in the range of 0.5 mm to 5 mm or in the range of 1 mm to 5 mm or in the range of 1 mm to 3 mm. A maximum adjustment of up to 3 mm may be practically sufficient. Values below 0.5 mm may be used if e.g. a stiff material is used.

However, a distance D4B between these two parts 434, 403a is still 0 mm since spacer 450 is still outside of main housing part 402. Apertures 444, 445 and 446 are arranged on the same axial position of longitudinal axis A as is illustrated in FIG. 4B. This axial position is proximal of the position of an abutting element comprising a distally facing abutting face 465 that holds proximal end of a spring 460. Thus, spring 460 is not in the way of spacer 450. Alternatively, other design options may be chosen to prevent interference of a spring for biasing needle shroud 408 and spacer 450, e.g. usage of several spring elements placed laterally of apertures 444 to 446, e.g. out of the way that is used for insertion of spacer 450, see e.g. FIG. 5A springs 561, 562 and abutting faces 566a and 567a.

The abutting element may extend circular around longitudinal axis A. Alternatively, several abutting elements may be used. The distal end of spring 460 may press against a radially inwardly directed ledge of distal portion 408a. Spring 460 may be a compression spring, e.g. made of metal. Spring 460 may bias distal portion 408a of needle shroud 408 in the distal D direction.

As is also apparent from FIG. 4B, arms 408b1 and 408b2 of needle shroud 408 are not located in the illustrated longitudinal cross section and are therefore not in the way of spacer 450.

As spacer 450 is not inserted, i.e. it is in a non-used or inactivated state, drug delivery device 400 has a maximum insertion depth and/or injection depth.

As is illustrated in FIG. 4C, bifurcated spacer 450 may comprise:
A basis portion 452, e.g. essentially of a rectangular shape,
A first prong 454, e.g. an elongated straight prong 454,
A second prong 456, e.g. an elongated straight prong 456.

Prongs 454 and 456 may extend parallel to each other from basis portion 452. There may be an opening 458 (intermediate space) between both prongs 454 and 456. As already mentioned ramps R may be located on the free ends of spacer 450. A lateral width Wi2 of bifurcated spacer 450 may be slightly smaller than lateral width Wi1 of apertures 444, 445 and 446 that are configured to receive bifurcated spacer 450.

FIG. 4C illustrates a transversal cross section through drug delivery device 400 with bifurcated spacer 450 laterally inserted into device 400. As is clearly visible, neck portion 436 is positioned between prongs 454 and 456 in intermediate space 458.

FIG. 4D illustrates a longitudinal cross section of drug delivery device 400 in the state according to the FIG. 4C, i.e. in the state in which spacer 450 is inserted into device 400. Prongs 454 and 456 of spacer 450 are arranged between shoulder 434 and proximal facing face of bottom portion 403a, see distance D4D. Distance D4D is greater than distance D4B.

Thus, syringe 430 is displaced proximally by a value that is equal to thickness T1 of spacer 450. Displacement of syringe 430 displaces needle 410 by the same amount proximally. The injection depth in the state of spacer 450 illustrated in FIG. 4D is therefore smaller or reduced compared to the injection depth in the inactivated state of spacer 450, e.g. state illustrated in FIG. 4B.

A resilient element may be used that is similar to resilient element 212, e.g. biasing syringe 430 distally and providing resiliency that enables axial movement of syringe 430 in the proximal P direction.

FIG. 5A illustrates a longitudinal cross section of a drug delivery device 500 according to a fourth embodiment. Drug delivery device 500 may comprise:
A main housing part 502, e.g. similar to housing 102, 202, 402, etc.,
A central part 503 of main housing part 502, e.g. similar to central part 403 but e.g. without a bottom portion 403a but comprising a distal rim or distal arms extending inwardly, e.g. closer to longitudinal axis A,
An actuating element 508, e.g. needle shroud 508, e.g. similar to needle shroud 208, 408, etc.,
A needle 510, e.g. similar to needle 410, and
A syringe 530.

Furthermore, drug delivery device 500 may comprise all or some of the parts mentioned above for drug delivery device 100, 200, etc., e.g. a piston rod, a drive mechanism, etc. Apertures 544, 545 and 546 may correspond to apertures 444, 445 and 446 respectively.

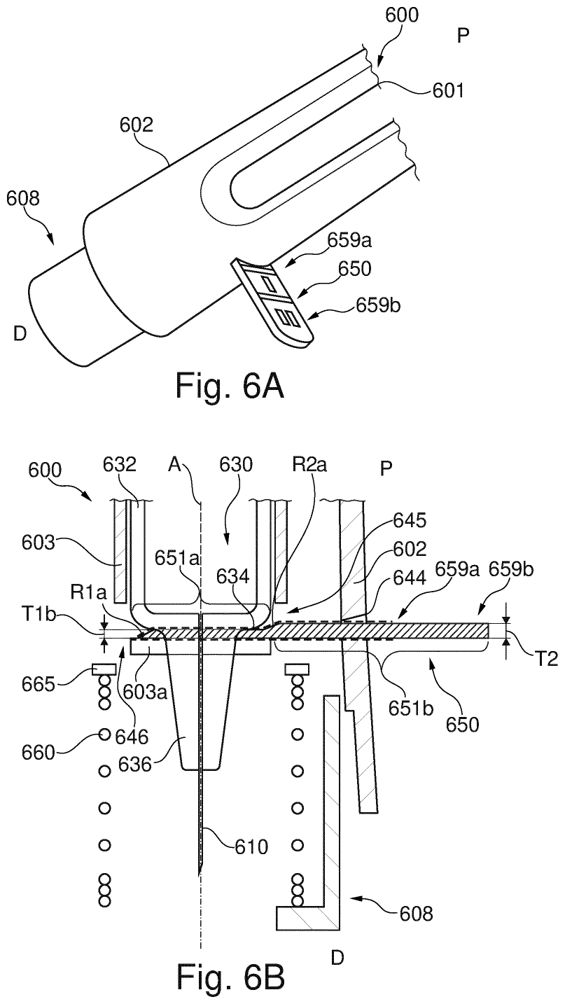

A surface of a skin 520 is illustrated. Device 500 is pressed with its needle shroud 508 against skin 520 in order to inject drug Dr. Depending on whether spacer 450 (only one thickness) or spacer 650 (different thicknesses) is used or not, it is possible to adjust at least two injection depths or three different injection depths in the case that spacer 650 is removable, see description of FIGS. 4a to 4D as well as description of FIGS. 6A and 6B.

Syringe 530 may comprise a distal portion 536 (neck, cone), a shoulder 534 and a barrel 532 as well as an optional proximal flange portion. Syringe 530 may comprise e.g. 2 ml (milliliter) of drug Dr solution if filled completely or almost completely.

In the fourth embodiment, more than one compression spring may be used to bias needle shroud 508 distally, e.g. a first compression spring 561, a second compression spring 562 and one or more optional further compression springs (not shown). First compression spring 561, second compression spring 562, etc. may have diameters that are much less compared to the diameter of distal portion 508a of needle shroud 508, e.g. less than 20 percent or less than 10 percent of the diameter of distal portion 508a of needle shroud 508. Compression springs 561, 562, etc. may be arranged near the inner surface of the distal portion of needle shroud 508 and at locations such that they do not interfere with spacer 450, 650, especially if more proximal abutting faces 566a and 567a are used as is explained in the following in more detail.

Instead of only one continuous abutting face, e.g. 465, several abutting faces may be used, e.g.:
An abutting face 566 for proximal end of first spring 561,
An abutting face 567 for proximal end of second spring 562, and
Optional further abutting faces for further springs.

Abutting faces 566, 567, etc. may be arranged at the same face or surface, e.g. on a circumferential rim, a circumferential ledge, etc. Alternatively, different protrusions or holes may be used to provide proximal holding for springs 561, 562, etc. Abutting faces 566, 567, etc. are arranged distally of spacer 450, 650 if spacer 450, 650 is within device 500. If spacer 450, 650 is not in place, the receiving space of spacer 450, 650 may be used as a reference, e.g. abutting faces 566, 567, etc. are arranged distally of a receiving space for spacer 450, 650.

Usage of a plurality of springs allows to place abutting faces 567a, 566a that correspond to abutting faces 566, 567 proximal of the receiving space of spacer 450, 650 and/or of spacer 450, 650. Appropriate placement of springs 561, 562, etc. may ensure that spacer 450, 650 is insertable without touching one of the springs 561, 562. On the other hand, it may be ensured that springs 561 and 562 do not touch spacer 450, 650 if springs 561, 562 are compressed or released during or after injection.

However, alternatively, it is of course possible to use only one compression spring, e.g. similar to compression spring 560, in order to bias needle shroud 508 distally.

Spacer 450 provides a distance D5A between a proximal facing face of distal arms or distal rim of central part 503 and shoulder 534. Distance D5A may be the same distance as a distance D5B if the same spacer 450, 650 or spacers of the same thickness are used. Distance D5A may decrease the injection depth of device 500 by a value that is equal to the value of distance D5A compared to the case in which spacer 450 is not used in device 500.

Again, a resilient element may be used that is similar to resilient element 212, e.g. biasing syringe 530 distally and providing resiliency that enables axial movement of syringe 530 in the proximal P direction.

FIG. 5B illustrates a longitudinal cross section of a drug delivery device according to a fifth embodiment. Drug delivery device 500x may comprise:
A main housing part 502x, e.g. similar to housing part 502,
A central part 503x, e.g. similar to central part 503,
An actuating element 508x, e.g. similar to needle shroud 508x,
A needle 510x, e.g. similar to needle 510,
A syringe 530x, e.g. similar to syringe 530, and
A syringe carrier 580 described below in more detail.

Furthermore, drug delivery device 500x may comprise all or some of the parts mentioned above for drug delivery device 100, 200, etc., e.g. a piston rod, a drive mechanism, etc. Apertures 544x, 545x and 546x may correspond to apertures 444, 445 and 446 respectively.

A surface of a skin 520x is illustrated. Device 500x is pressed with its needle shroud 508x against skin 520x in order to inject the drug. Depending on whether spacer 450 (only one thickness) or spacer 650x (different thicknesses) is used or not, it is possible to adjust at least two injection depths or three injection depths in the case that spacer 650 is removable, see description of FIGS. 4a to 4D as well as description of FIGS. 6A and 6B.

Syringe 530x may comprise a distal portion 536x (neck, cone), a shoulder 534x and a barrel 532x as well as an optional proximal flange portion. Syringe 530x may comprise e.g. 1 ml of drug Dr, e.g. a solution, if filled completely or almost completely.

In the fifth embodiment only one compression spring 560x may be used in order to bias needle shroud 508x distally. An abutting face 565 for holding proximal end of spring 560x may be arranged distally with respect to spacer 450, 650 and/or to a retaining space for spacer 450, 650.

It is of course possible to use alternatively several compression springs, see 561, 562 in FIG. 5A. If several compression springs are used, it is possible to have abutting faces for proximal ends of these springs 561, 562 which are arranged distally or proximally of spacer 450, 650 and/or relative to a retaining space for a spacer, e.g. 450, 650.

Syringe carrier 580 may comprise:
At least one distal arm 582, 584, alternatively a circular rim or ledge,
A barrel like main housing, e.g. comprising one or two drug window(s), and/or
An optional proximal flange portion, and/or
At least one hook on flange portion or elsewhere allowing axial fixation of the syringe carrier within the housing 502x.

There may be an optional distance between optional flange portion of carrier 580 and an optional flange of syringe 580. In other embodiments, a spacer may be used to enlarge this distance in order to provide needle injection depth adjustment.

However, in the fifth embodiment, a spacer, e.g. 450, 650, is inserted between the distal ends of arms 584 and a proximal facing face of distal arms or distal rim of central part 503x and or between shoulder 534x and a proximal facing face of distal arms or distal rim of central part 503x. Syringe 530x and syringe carrier 580 are moved proximally by the spacer, e.g. 450, 650 by a distance D5B. Distance D5B may decrease the injection depth by a value that is equal to the value of distance D5B compared to the case in which no spacer is used, e.g. none of the spacers 450, 650.

A resilient element may be used that is similar to resilient element 212, e.g. biasing syringe 530x distally and providing resiliency that enables axial movement of syringe 530x in the proximal P direction.

The principles that are described below for pivotable spacers or for cam spacer elements may also be applied in both cases, e.g. in cases without a separate carrier for the container or in cases with a separate carrier 580 for the container. Pivotable spacer elements or pivotable cam spacer elements (cams) may interfere at the same locations that are illustrated in FIGS. 5A and 5B and/or that are described in the description of FIGS. 5A and 5B, e.g. on opposite sides of the carrier/syringe. In all cases using pivotable spacer elements/cams, only one compression spring, e.g. 560x may be used to bias the needle shroud distally, preferably using abutting face(s) for the proximal end of this compression spring located distally from spacer elements/cams or from a retaining space for the spacer elements/cams. Alternatively, in all cases using pivotable spacer elements/cams, more than one compression spring, e.g. 561, 562 may be used to bias the needle shroud distally, wherein abutting face(s) for the proximal end of these compression spring(s) may be located distally or proximally from spacer elements/cams or from a retaining space for the spacer elements/cams.

FIG. 6A illustrates a perspective view of a drug delivery device 600 according to a sixth embodiment configured to receive a bifurcated spacer element 650 comprising two different spacer thicknesses. Drug delivery device 600 may comprise:
At least one optional drug window 601 or at least two drug windows for monitoring injection, e.g. arranged within the surface of a main housing part 602,
A main housing part 602, e.g. similar to main housing parts 102, 202, etc.,
An actuating element 608, e.g. needle shroud 608, and
Further parts that are mentioned below and illustrated in FIG. 6B.

A bifurcated spacer element 650 may comprise two thicknesses T1b, T2, see FIG. 6B. As is illustrated in FIG. 6A, there are e.g. two identifiers on an operating portion of spacer element 650, e.g. a first identifier 659a, e.g. a “I” (Roman one) indicating that the first thickness T1b is used or active, and a second identifier 659b, e.g. a “II” (Roman two) indicating that second thickness T2 is used. The reference for identifiers 659a, 659b may be main housing part 602 and/or lines near the identifier 659a, 659b aligned with main housing part. In the state that is illustrated in FIG. 6A, spacer 650 is in a first position that corresponds to identifier “I”, i.e. the first thickness T1b is active in more detail below, see FIG. 6B. If spacer element 650 is inserted deeper into device 600 the second thickness T2 becomes active as is also described in more detail below, see FIG. 6B.

Bifurcated spacer element 650 may be a built-in component of device 600, e.g. it may not be possible to remove spacer element 650 out of device 600. Alternatively, spacer element 650 may be removable from device 600.

FIG. 6B illustrates a longitudinal section of drug delivery device 600 according to FIG. 6A. Drug delivery device 600 may further comprise:
A central part 603 (central portion) of main housing part 602, e.g. similar to central part 403,
A needle 610, e.g. similar to needle 110, 210, etc., and
A syringe 630.

A bottom 603a of central part 603 may be used to define a surface on which syringe 630 is positioned axially.

Syringe 630 may comprise a barrel 632, a shoulder 634 and a distal portion 636 (neck, cone).

Furthermore, drug delivery device 600 may comprise all or some of the parts mentioned above for drug delivery device 100, 200, etc., e.g. a piston rod, a drive mechanism, etc. Apertures 644, 645 and 646 may correspond to apertures 444, 445 and 446 respectively.

As is illustrated in FIG. 6B, bifurcated spacer element 650 has two thicknesses T1b, T2. A first thickness portion 651a (region) has thickness T1b (height), e.g. a thickness in the range of 0.5 mm to 3 mm. A second thickness portion 651b (region) may have a thickness T2 that is greater than thickness T1b, e.g. by at least 30 percent or by at least 50 percent of thickness T1b. The operating portion of spacer 650 may also have thickness T2 or a different thickness, e.g. again thickness T1b. The operating portion may comprise the first identifier 659a and the second identifier 659b. Alternatively, the operating portion may be a separate portion that does not carry any identifiers.

In another embodiment, there may be more than two portions of different thickness, e.g. three or four portions, on bifurcated spacer element 650.

As is apparent from FIG. 6B, only one compression spring 660 may be used to bias needle shroud 608 distally. An abutting face 665 may be arranged distal of spacer 650 and/or of a retaining space for spacer 650 defined by the apertures 644 to 646.

A first ramp R1a may be arranged on the free end of a first prong of spacer 650. A second ramp R1a may be arranged on the free end of a second prong of spacer 650. Ramps R1a and R1b may be inclined to the free ends of the first prong and of the second prong in order to ease insertion of spacer 650 between shoulder 634 and proximal face of bottom portion 603a using e.g. the force enhancing properties of a wedge. In a similar manner, ramps R2a, R2b may be arranged between first thickness portion 651a and second thickness portion 651b in order to ease further insertion of spacer 650, e.g. changing from first state to second state.

Syringe 630 may be displaced proximally P by first thickness portion 651a by a first displacement length that corresponds, e.g. is equal to, thickness T1b thus reducing the injection depth of device 600 compared to the injection depth with no spacer present or defining a first injection depth in case that spacer 650 is not removable from device 600. Moreover, syringe 630 may be displaced proximally P by second thickness portion 651b in a second state (position) of spacer 650 by a second displacement length that corresponds (is equal to) thickness T2 thus reducing the injection depth of device 600 further compared to the injection depth in the first state of spacer 650. Thus, device 600 may be appropriate for administering drugs Dr to children (first state and second state) and to adults (no spacer 650 present) if spacer 650 is removable. If spacer 650 is a built-in spacer, the first state (position) of spacer 650 may be used for administering drugs Dr to adults and the second state (position) of spacer 650 may be used for administering drugs Dr to children.

A resilient element may be used that is similar to resilient element 212, e.g. biasing syringe 630 distally and providing resiliency that enables axial movement of syringe 630 in the proximal P direction.

It is of course possible to use spacer element 650 in the fourth embodiment, see FIG. 5A (drug delivery device without separate syringe carrier) or in the fifth embodiment, see FIG. 5B (drug delivery device with separate syringe carrier 580).

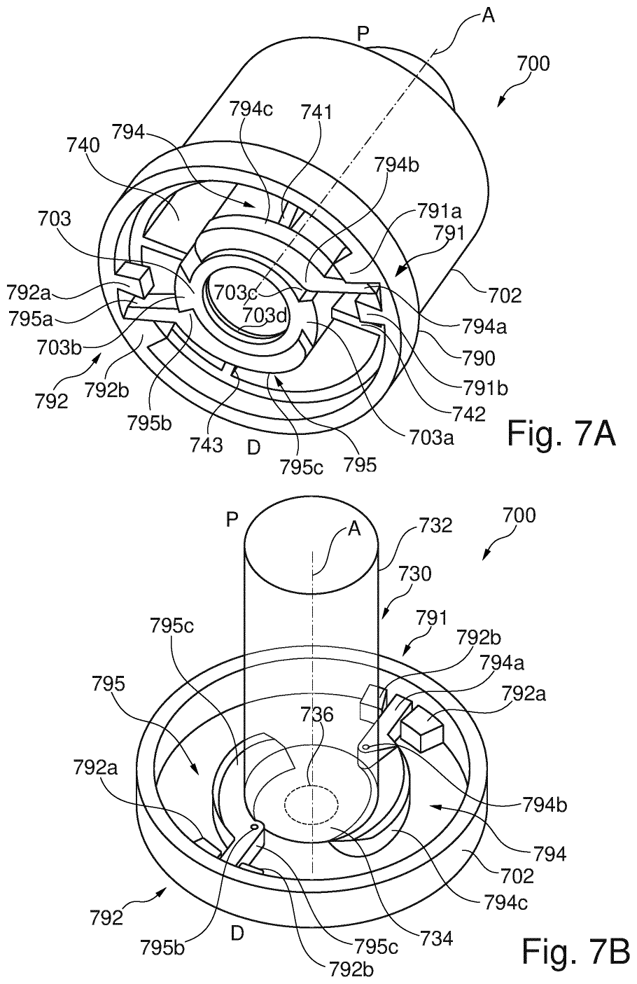

FIG. 7A illustrates a perspective view of a drug delivery device 700 according to a seventh embodiment comprising two pivotable spacer arms 794c and 795c, e.g. mounted on first class levers 794 and 795. Drug delivery device 700 may comprise in addition to levers 794 and 795:
A main housing part 702, e.g. similar to main housing part 102, 202, etc.,
A ring 790 enabling operation of levers 794 and 795, e.g. adjustment of the pivoting angle, by a user of device 700, and
A syringe 730, see FIG. 7B.

Furthermore, drug delivery device 700 may comprise all or some of the parts mentioned above for drug delivery device 100, 200, etc., e.g. a piston rod, a drive mechanism, a needle shroud, etc.

Main housing part 702 may comprise a central part 703 (portion). Central part 703 may be hold by axial/radial ribs 740 to 744. Furthermore, radial protrusions 703a, 703b extend from central part 703 (portion) radially outwards providing an intermediate space for the arrangement of spacer arms 794 and 795. Moreover, apertures 703c, 703d, e.g. slits, are formed within central part 703 in order to enable insertion of spacer arms 794c and 795c through central part 703 into the border between a shoulder 734 of syringe 730 and a distal surface (proximally facing) of aperture 703c or 703d.

Central portion 703 may comprise a rim or a ledge protruding radially inwards in order to position syringe 730 in the axial direction. Alternatively, distal arms may be used on central portion 703 to fulfill the same function.

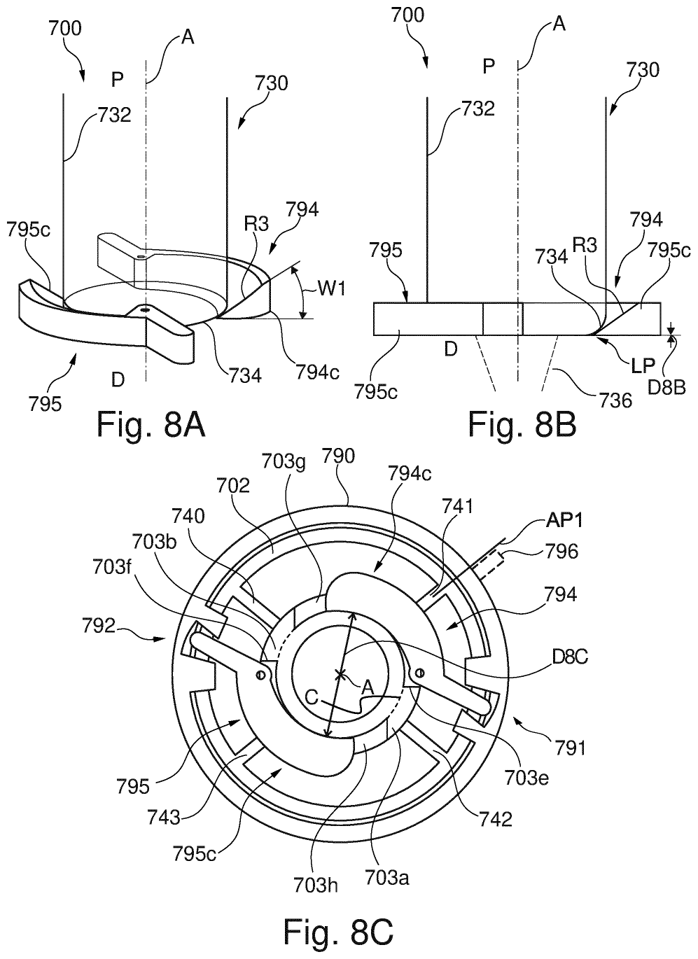

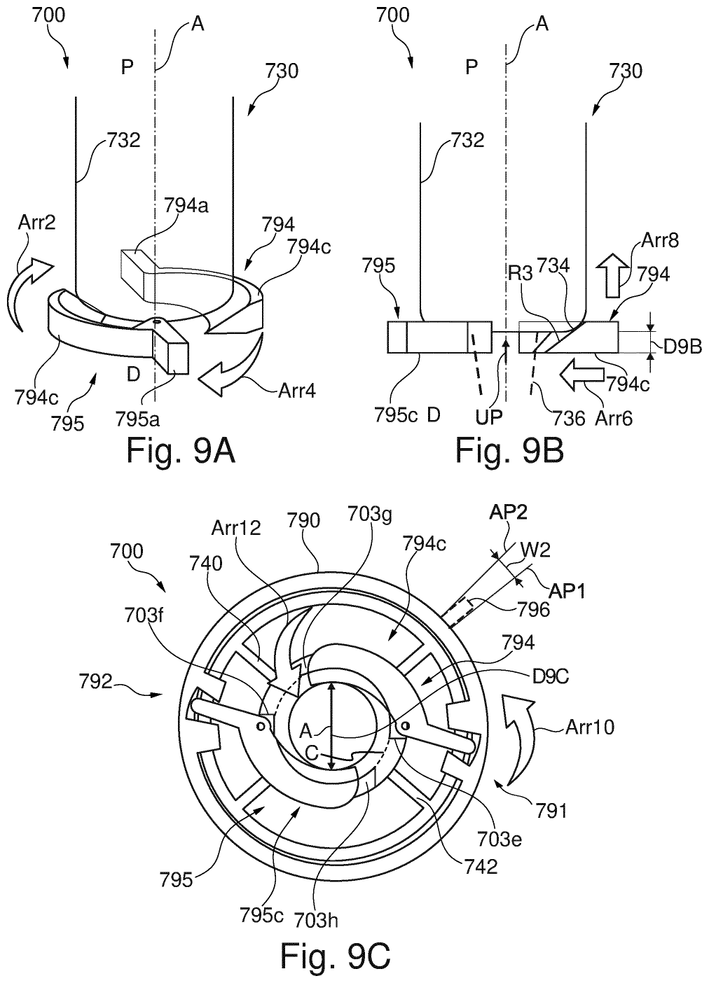

In the embodiment there may be the following faces on protrusions 703a, 703b, see FIGS. 8C and 9C:
Stop faces 703e, 703f, e.g. configured to stop rotation of ring 790 in one rotation direction, and
Slanted faces 703g, 703h, e.g. configured to promote insertion of spacer arms 794c and 795c.

Syringe 730 may comprise a barrel portion 732, a shoulder 734 and a distal portion 736 (neck, cone). Furthermore, syringe 730 may comprise a proximal flange (not shown). Alternatively, a container may be used comprising a distal attachment portion for a needle, a neck portion, and a barrel portion. There may be no proximal flange on this container.

A first ring/lever interface 791 may comprise two protrusions 791a, 791b arranged on the inside of ring 790 and forming a first intermediate space between both protrusions 791a, 791b. An operating portion 794a of lever 794 may be arranged within this first intermediate space allowing mechanical contact between operating portion 794a and protrusions 791a, 791b thereby transmitting and/or converting rotation movement of ring 790 into a pivoting movement of lever 794. In a similar way, a second ring/lever interface 792 may comprise two protrusions 792a, 792b arranged on the inside of ring 790 opposite to interface 791 and forming a second intermediate space between both protrusions 792a, 792b. An operating portion 795a of lever 795 may be arranged within this second intermediate space allowing mechanical contact between operating portion 795a and protrusions 792a, 792b thereby transmitting and/or converting rotation movement of ring 790 into a pivoting movement of lever 795.

First lever 794 may be a first class lever, e.g. a lever comprising two lever arms. Lever 794 may comprise:
An operating portion 794a, e.g. an elongated and/or straight portion,
A hinge portion 794b (pivoting point), e.g. arranged between operating portion 794a and spacer arm 794c, and
A spacer arm 794c, e.g. a spacer arm comprising a ramped profile and/or a curved spacer arm that is curved in circumferential direction of housing 702.

Second lever 795 may also be a first class lever, e.g. a lever comprising two lever arms. Lever 794 may comprise:
An operating portion 795a, e.g. an elongated and/or straight portion,
A hinge portion 795b (pivoting point), e.g. arranged between operating portion 795a and spacer arm 795c, and
A spacer arm 795c (ramped profile), e.g. a spacer arm comprising a ramped profile and/or a curved spacer arm that is curved in circumferential direction of housing 702.

First lever 794 and second lever 794 may have identical shapes enabling simplified logistics.

FIG. 7B illustrates a perspective view from above and from the front to the drug delivery device according to FIG. 7A. Syringe 730 is illustrated. However, central part 703 is omitted in order to give a free view to the interference of spacer arms 794c and 795c and shoulder 734.

FIG. 8A illustrates a perspective view of device 700 with levers 794, 795 in an inactivated first position (state), e.g. spacer arms 794c and 795c interfere only slightly or do not interfere with shoulder 734. As is apparent from FIG. 8A, a ramp R3 is formed on spacer arm 794c. Ramp R3 is inclined such that the height of ramp R3 increases continuously as the distance to longitudinal axis A increases when moving radially outwards. There may be a region of constant thickness adjacent to and radially outwards of ramp R3. Within a cross section that includes the longitudinal axis A, there may be an angle W1 of ramp R3. Angle W1 may be in the range of 10 degrees to 45 degrees, preferably within the range of 20 degrees to 40 degrees. A similar ramp may be formed on spacer arm 795c.

FIG. 8B illustrates a side view of device 700 with the levers 794, 795 and spacer arms 794c, 795c in the inactivated first position (state). As is apparent from FIG. 8B, syringe 730 is still in a lower position LP as a distance D8B between a distal surface of the bottom of barrel 732 (distal end of shoulder 734) and a distal face of spacer arm 794c (first state) is 0 mm, e.g. syringe 730 is not moved proximally P by ramp R3 and corresponding ramp on spacer arm 795c. Distal face of spacer arm 794c may have essentially the same axial position as a distal face of aperture 703c. Distal face of spacer arm 795c may have essentially the same axial position as a distal face of aperture 703d.

FIG. 8C illustrates a bottom view of device 700 with spacer arms 794c, 795c in the inactivated position (state). A momentary minimum radial distance D8C (first state) between levers 794 and 795, more exactly between spacer arms 794c, 795c, including longitudinal axis A has a value that corresponds essentially to the outer diameter of syringe barrel 732, see circle C. The difference between distance D8C and outer diameter of barrel 732 may be less than 10 percent or less than 5 percent of the outer diameter of barrel 732. Thus, there is no overlap or only a slight overlap of barrel 732 and spacer arms 794c, 795c as viewed in direction of longitudinal axis A, see FIG. 8C.

As illustrated in FIG. 8C, an optional operating protrusion 796 may be arranged on the outside of ring 790 in order to ease operating ring 790, e.g. rotating. Alternatively, more than one protrusion 796 may be arranged on the outside of ring 790, e.g. arranged equidistantly to each other. Further, alternatively, ribs or other gripping means may be arranged on the outer surface of ring 790 in order to ease operation, e.g. rotation of ring 790. It is possible to use a knurled outer surface on ring 790. Furthermore, ring 790 may protrude out of housing 702 radially outwards, e.g. along the whole circumference of housing 702.

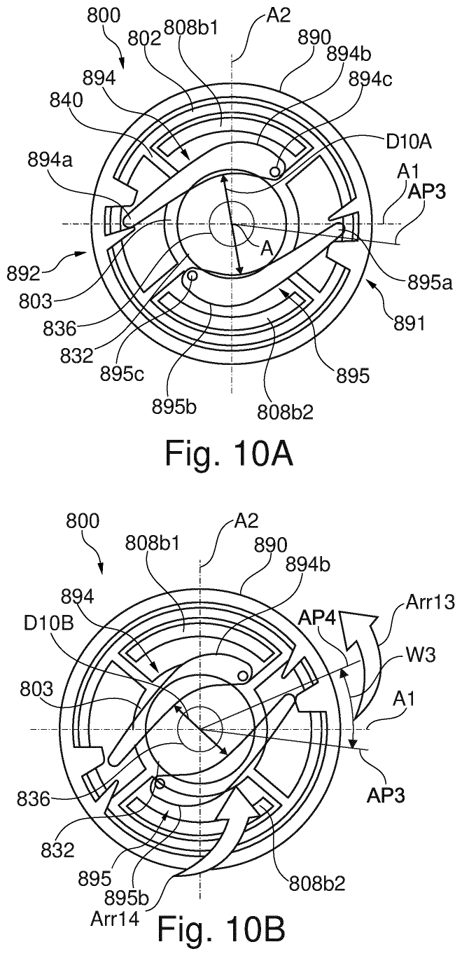

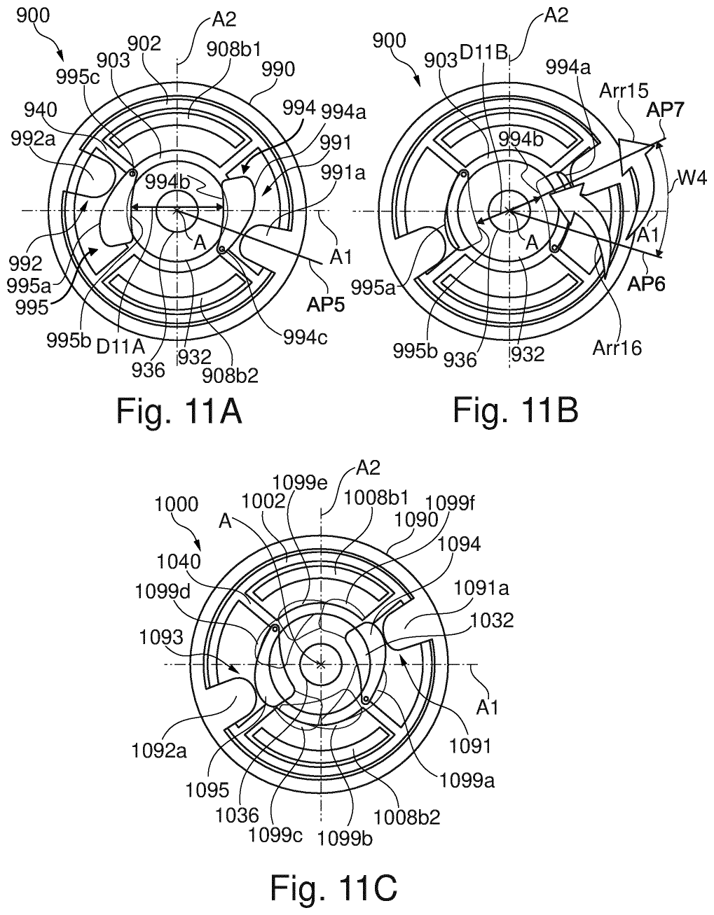

According to FIG. 8C there is a first angular position AP1 of ring 790 in the first state, see e.g. “front” (e.g. with regard to a rotation direction) face of optional protrusion 796. Needle shroud arms are omitted in FIG. 8C. However, FIGS. 10A to 11C illustrate the arrangement of needle shroud arms between ribs that correspond to ribs 740 and 741 as well as between ribs that correspond to ribs 742 and 744. Thus, interfaces 791 and 792 do not interfere with the arms of the needle shroud.

According to FIGS. 8A to 8C there is no interfering or only little interfering of spacer arms 794a, 794b and barrel 732 through apertures 703c, 703d in the first state (position) of spacer arms 794a, 794b. Barrel 732 is not visible in FIG. 8C. However, circle C indicates the outer circumference of barrel 732.

Moreover, as is described in more detail in the following, according to FIGS. 9A to 9C there may be much more interference of spacer arms 794c, 795c and barrel 732 in a second state (position) of spacer arms 794c, 794c.

FIG. 9A illustrates a perspective view of device 700 with spacer arms 794c, 795c in an activated second position (state). Arrow Arr2 indicates a clockwise movement of operating portion 794a if ring 790 is rotated in the clockwise direction. In the same way, arrow arr4 indicates a clockwise movement of operating portion 795a if ring 790 is rotated in the clockwise direction. The clockwise movements of operating portions 794a, 795a are converted by levers 794 and 795 in radially inwardly directed movements of spacer arms 794c, 795c.

FIG. 9B illustrates a side view of device 700 with spacer arms 794c, 795c in the activated second position (state). As is apparent from FIG. 9B, the radially inwardly directed movements of spacer arms 794c, 795c moves syringe 730 proximally to an upper position UP. An arrow Arr6 indicates the radial inwards directed movement of spacer arm 794c. An arrow Arr8 indicates the proximal movement of syringe 730 whereby shoulder 734 slides upwards on ramp R3.

At the end of the proximal movement of syringe 730, a distance D9B between the distal end of shoulder 734 and the distal face of spacer arm 794c is in the second state greater than distance D8B, see FIG. 8B. Distance D9B may be in the range of 0.5 mm to 5 mm or in the range of 1 mm to 3 mm. Distance D9B may be equal to thickness or axial height of spacer arm 794c or 795c. Alternatively, distance D9B may be e.g. in the range of 80 percent of distance D9B to 100 percent or 95 percent of distance D9B in the second state.

FIG. 9C illustrates a bottom view of device 700 with spacer arms 794c, 795c in the activated second position. A distance D9C is a momentary minimum radial distance between levers 794 and 795, more exactly between spacer arms 794c, 795c including longitudinal axis A. As is apparent from FIG. 9C, there is an overlap between spacer arm 794c and barrel 732 as well as between spacer arm 795c and barrel 732 as viewed in the direction of longitudinal axis A. Barrel 732 is not visible in FIG. 9C. However, circle C indicates the outer circumference of barrel 732.

In the second state of levers 794, 795, minimum radial distance D9C is less than minimum radial distance D8C that is valid for the first state, see FIG. 8C. Distance D9C may e.g. be less than 90 percent or less than 80 percent of distance D8C.

Again, needle shroud arms are omitted in FIG. 9C. However, needle shroud arms are positioned on corresponding places to the places shown e.g. for needle shroud arms 808b1 and 808b2 in FIG. 10A. Thus, there is no interference between needle shroud arms and operating portion 794a of lever 794 and operating portion 795a of lever 795.

An angle W2 is illustrated between the first angular position AP1 and a second angular position AP2 to which ring 790 has been rotated in a counterclockwise direction (this results from the bottom view but confirms to the clockwise rotation mentioned in the description of FIG. 9A), see e.g. “front face of optional protrusion 796. Rotation of ring 790 is indicated by an arrow Arr10. Rotation of ring 790 is transmitted to operating portions 794a, 795a via interface 791 and 792 respectively. Lever 794 converts counterclockwise movement of its operating portion 794a into a radial inwards directed movement of spacer arm 794c, see arrow Arr12. In a similar way, lever 795 converts counterclockwise movement of its operating portion 795a into a radial inwards directed movement of spacer arm 795c.

A resilient element may be used that is similar to resilient element 212, e.g. biasing syringe 730 distally and providing resiliency that enables axial movement of syringe 730 in the proximal P direction.

Thus, FIGS. 9A to 9C illustrate a considerable interfering of spacer arms 794c, 795c and barrel 732 through apertures 703c, 703d. This interference may be used to adjust the injection depth, preferably to reduce the injection depth of device 700. A reduced injection depth may e.g. have advantages for administering drugs Dr to children.

FIG. 10A illustrates a bottom view of a drug delivery device 800 according to an eighth embodiment comprising two pivotable levers 894 and 895 in an inactivated first position (state). Drug delivery device 800 may comprise in addition to levers 894 and 895:
A main housing part 802, e.g. similar to main housing part 102, 202, etc.,
A central part 803, e.g. similar to central part 203, 403, etc.,
Four axial/radial ribs holding central part 803 within housing part 802 coaxially, see e.g. axial/radial rib 840,
A needle shroud (not shown) that may be named as activating element or as needle protection element, see arms 808b1, 808b2 of needle shroud (not shown),
A syringe (not shown), see barrel 832 and distal portion 836 (neck, cone) of the syringe, and
A ring 890 for operating levers 894 and 895.

Furthermore, drug delivery device 800 may comprise all or some of the parts mentioned above for drug delivery device 100, 200, etc., e.g. a piston rod, a drive mechanism, etc.

A first ring/lever interface 891 may comprise two protrusions arranged on the inside of ring 890 and arranged with an angular distance greater 0 mm to each other forming a first intermediate space. A second ring/lever interface 892 may be arranged on ring 890 on a side that is opposite to the side on which interface 891 is arranged. The second ring/lever interface 892 may comprise two protrusions arranged on the inside of ring 890 and arranged with an angular distance greater 0 mm to each other forming a second intermediate space.

First lever 894 may be a second class lever, e.g. a lever comprising only one lever arm. Lever 894 may comprise:
An operating end 894a arranged within the first intermediate space,
A spacer arm 894b arranged between operating end 894a and a hinge portion 894c, and
A hinge portion 894c (pivoting point) at the other end of first lever 894.

Second lever 895 may also be a second class lever, e.g. a lever comprising only one lever arm extending only to one side from the pivoting end. Lever 895 may comprise:
An operating end 895a, arranged within the second intermediate space,
A spacer arm 895b arranged between operating end 895a and a hinge portion 895c, and
A hinge portion 895b (pivoting point) at the other end of second lever 895.

There is a momentary minimum radial distance D10A in the first state (position) between levers 894, 895, especially between spacer arms 894b and 895b. Spacer arms 894b and 895b do not interfere or interfere only slightly with barrel 832 of the syringe. Thus, the syringe is in its most distal position and the injection depth has a maximum. The maximum injection depth may be appropriate to administer drugs Dr to adults. Furthermore, ring 890 is in a third angular position AP3 in the first state, see e.g. front edge of the second protrusion of interface 891.

FIG. 10B illustrates a bottom view of the device according to FIG. 10A with spacer arms in an activated second position (state). Ring 890 may have been rotated by a user to a fourth angular position AP4. An angle W3 and an arrow Arr13 indicate this rotation. This rotation transfers to a radial inwards movement of spacer arms 894b and 895b of levers 894 and 895, see arrow Arr14.

There is a momentary minimum radial distance D10B in the second state (position, configuration) between levers 894, 895. Distance D10B is less than distance D10A, see FIG. 10B, e.g. less than 90 percent or less than 80 percent of distance D10A. Therefore, there is a considerable interference or overlap of spacer arms 894b and 895b with barrel 832 as seen in the direction of the longitudinal axis A. The syringe, e.g. barrel 832 as well as neck portion 836 are moved proximally by this interference. Therefore, the injection depth is reduced. A reduced injection depth may be appropriate for administering drugs Dr to children.

FIGS. 10A and 10B illustrate second class levers 894, 895 that may have a better transmission of force from ring 890 via operating end 894a, 895a to spacer arms 894b, 895b and finally to barrel 832 in order to initiate and to perform proximal translation of the syringe.

In another embodiment, ring 890 may carry at least one protrusion, rib or other means (e.g. knurled surface, radial protruding ring) on its outside in order to ease rotation of ring 890 by a user, see e.g. protrusion 796 as illustrated in FIGS. 8C, 9C.

As is also apparent from FIGS. 10A and 10b there is no interference between interfaces 891, 892 and arms 808b1, 808b2 of the needle shroud enabling undisturbed operation of all four elements.

FIG. 11A illustrates a bottom view of a drug delivery device according to a ninth embodiment comprising two cam spacer elements 994, 995 being in an inactivated first position (state). Drug delivery device 900 may comprise:
A main housing part 902, e.g. similar to main housing part 102, 202, etc.,
A central part 903 (portion), e.g. similar to central portion 203, 403, etc.,
Four axial/radial ribs holding central part 903 within housing part 902 coaxially, see e.g. axial/radial rib 940,
A needle shroud (not shown) that may be named as activating element or as needle protection element, see arms 908b1, 908b2 of needle shroud (not shown),
A syringe (not shown), see barrel 932 and distal portion 936 (neck, cone) of the syringe, and
A ring 990 for operating cam spacer elements 994, 995.

Furthermore, drug delivery device 900 may comprise all or some of the parts mentioned above for drug delivery device 100, 200, etc., e.g. a piston rod, a drive mechanism, etc.

A first cam/protrusion interface 991 may comprise a protrusion 991a arranged on the inside of ring 990 and may be configured to make continuous and steady contact with an outer cam surface 994a of cam spacer element 994. A second ring/lever interface 992 may be arranged on ring 990 on a side that is opposite to the side on which interface 991 is arranged. The second cam/protrusion interface 992 comprises a single protrusions 992a arranged on the inside of ring 990 and may be configured to make continuous and steady contact with an outer cam surface 995a of cam spacer element 995.

First cam-spacer element 994 may comprise:
An outer cam surface 994a, e.g. a curved surface 994a or face,
An inner ramped portion 994b, e.g. a curved portion 994b, and
A hinge portion 994c (pivoting point) enabling pivoting of cam spacer element 994 as the protrusion 991a of interface 991 moves along outer cam surface 994a.

Second cam spacer element 995 may comprise:
An outer cam surface 995a, e.g. a curved surface 995a or face
An inner ramped portion 995b, e.g. a curved portion 995b, and
A hinge portion 995c (pivoting point) enabling pivoting of cam spacer element 995 as the protrusion 992a of interface 992 moves along outer cam surface 995a.

There is a fifth angular position AP5 if device 900 is in the first state (position) of cam spacer elements 994 and 995 as illustrated in FIG. 11A. Protrusion 991a may be at the fifth angular position in the first state.

As is further illustrated in FIG. 11A, a momentary minimum radial distance D11A may be established between the inner edges of inner ramped portions 994b and 995b in the first state. There, may be no axial overlap or only a slight axial overlap between inner ramped portions 994b, 995b and barrel 932 with respect to axis A, e.g. as viewed in the direction of longitudinal axis A. Thus, barrel 832 may abut to a bottom face of central part 903 or to another appropriate distal element of central part 903 as described in other embodiments above. This may mean that there is a maximum insertion depth that may be appropriate for administering drugs Dr to adults.

FIG. 11B illustrates a bottom view of device 900 according to FIG. 11A with the spacer elements 994, 995 in an activated second position (state). Ring 990 has been rotated by a user in counter clockwise direction to a sixth angular position AP6, see arrow Arr15. There is an angle W4 between fifth angular position AP5 and sixth angular position AP6. Due to this rotation of ring 990, protrusion 991a has moved along outer cam surface 994a into a region of cam spacer element 994 that has a greater radial width compared to the radial width of region(s) that are located closer to hinge 994c. Radial width of cam spacer element 994 may increase continuously as the distance of protrusion 991a to hinge portion 994 increases, e.g. up to a maximum width. There may be a slight decrease of radial width after reaching the maximum width,

Due to the greater radial width, cam spacer element 994a pivots and its free end is moved radially inwards bringing inner ramped portion 994b between barrel 932 and a proximal facing face of bottom portion of central portion 903 or of another appropriate distal structure of central portion 903, see arrow Arr16, thereby touching e.g. a shoulder of the syringe that is arranged between barrel 932 and neck portion 936. Similar operations may be true with regard to outer cam surface 995a and protrusion 992a resulting in a radial inwards movement of inner ramped portion 995b.

The syringe moves proximally since inner ramped portions 994b, 995b move inwardly. Finally, there is a momentary minimum radial distance D11B in the second state resulting in a most proximal position of the syringe. Distance D11B may be less than distance D11A, e.g. less than 90 percent or less than 80 percent of distance D11A. In the second state, injection depth of a needle of device 900 may be decreased compared to the injection depth in the first state. Thus, the second state of cam spacer elements 994, 995 may be appropriate to administer a drug Dr to a child.

If ring 990 is rotated back, cam spacer elements 994, 995 may move radially outwards due to the force of a resilient element acting on a proximal part of the syringe, e.g. on a flange of the syringe. The same may apply to all other embodiments mentioned in this description, e.g. to levers 794, 795, or 984, 895.

FIG. 11C illustrates a bottom view of a drug delivery device 1000 according to a tenth embodiment comprising more than two cam spacer elements 1094, 1095, 1099a to 1099f. Drug delivery device 1000 may essentially correspond to device 900, e.g.:
Main housing part 1002 may correspond to main housing part 902,
A central part (not visible) may correspond to central part 903.

Furthermore, drug delivery device 1000 may comprise all or some of the parts mentioned above for drug delivery device 100, 200, etc., e.g. a piston rod, a drive mechanism, etc.

The following elements of drug delivery device 1000 may correspond to elements of device 900 having a reference number that is decreased by value 100:
Arm(s) 1008b1, 1008b2 of needle shroud,
A distal portion 1036 of syringe (neck, cone), e.g. smaller diameter portion (neck) compared to barrel portion of syringe,
Axial/radial ribs, e.g. axial/radial rib 1040,
A ring 1090,
A first interface 1091 comprising a protrusion 1091a,
A second interface 1092 comprising a protrusion 1092a,
A first spacer element 1094, and
A second spacer element 1095.

There may be no further protrusions 1091a, 1092a assigned or allocated to further cam spacer elements 1099a to 1099f. Thus all cam spacer elements 1094, 1095, 1099a to 1099f may operate similar to an optical diaphragm with spacer elements 1094, 1095, 1099a to 1099f forming the blades of the diaphragm or of the diaphragm shutter. There, may be a plurality of cam spacer elements, e.g. within the range of 3 to 10 cam spacer elements operated by only two protrusions 1091a and 1092a. The radial length of a respective ramp of cam spacer elements 1094, 1095, 1099a to 1099f may be shorter compared to the radial length of the ramp on cam spacer elements 994 and 995 in order to ease or in order to enable appropriate interference of all cam spacer elements 1094, 1095, 1099a to 1099f. This interference may occur when closing the central opening formed by spacer elements 1094, 1095, 1099a to 1099f around neck portion 1036 of the syringe. Thus, spacer elements 1094, 1095, 1099a to 1099f may have also portions of constant thickness extending from the outward end of the “short” ramps to outer cam surfaces, e.g. corresponding to outer cam surface 994a, of cam spacer elements 1094, 1095, 1099a to 1099f.

Thus, again, the syringe may be displaced between at least two axial positions resulting in at least two different injection depths of device 1000. The smaller injection depth may for instance be more appropriate for administering drugs Dr to children compared to the larger injection depth.

For all embodiments described in the description of FIGS. 11A to 11C, a resilient element may be used that is similar to resilient element 212, e.g. biasing the syringe and providing resiliency that enables e.g. axial movement of the syringe in the proximal P direction and or outwards movement of spacer elements, e.g. 894c, 895c, 994, 995, 1094, 1095, 1099a to 1099f.

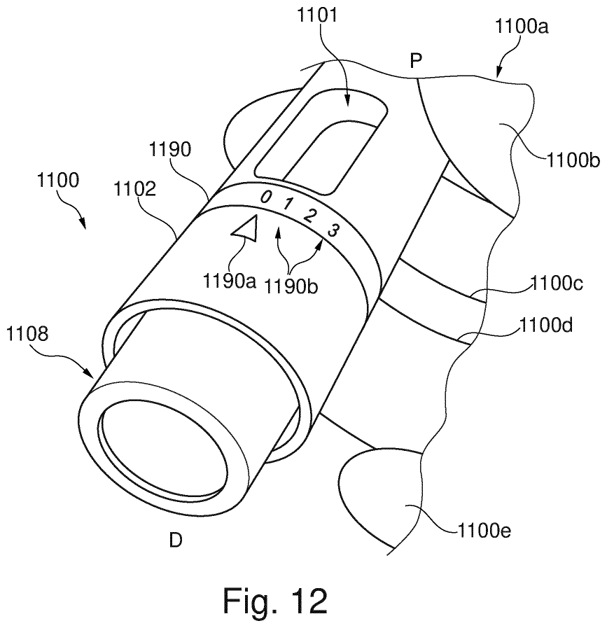

FIG. 12 illustrates a perspective view of a drug delivery device 1100 comprising a ring feature 1190, e.g. drug delivery device 700, 800, 900 or 1000. Drug delivery device 1100 may comprise:
An optional drug window 1101 or two optional drug windows,
A main housing part 1102, e.g. similar to housing parts 102, 202, etc. mentioned above,
A needle shroud 1108 (actuating element, needle protection element), e.g. similar to needle shroud 208, 408, etc. mentioned above, and
A ring 1190 for adjusting the injection depth of a needle (not shown) of device 1100.

Furthermore, drug delivery device 1100 may comprise all or some of the parts mentioned above for drug delivery device 100, 200, etc., i.e. a piston rod, a drive mechanism, etc.

Drug delivery device 1100 is hold by a hand 1100a, e.g. the hand of a child or of an adult who administers a drug to a child. Thumb 1100b, index finger 1100c, middle finger 1100d and ring finger 1100e of hand 110a are illustrated, e.g. thumb 1100b, index finger 1100c and middle finger 1100d may be used to administer drug Dr by pressing device 1100 against the skin (not shown) of the child. Similarly, e.g. thumb 1100b and index finger 1100c may be used to rotate ring 1190 in order to adjust the injection depth.

A marker 1190a may be arranged near ring 1190, e.g. distally of ring 1190. Marker 1190 may be e.g. an arrowhead, an arrow or another appropriate marker. Ring 1190 may comprise at least one identifier(s) 1190b, e.g. “0” indicating inactivated state of spacer elements, “1” indicating a first activated state, optionally further identifiers “2”, “3”, etc. indicating further states of spacer elements.

Ring 1190 may internally interact with at least one lever(s), with at least one cam element or with other appropriate spacer elements in order to adjust injection depth as described above in detail, see e.g. description of FIGS. 7A to 11C.

Spoken with other words, drug delivery device 100 to 1100 with adjustable injection depth may comprise:
A housing 102, 202, 402, etc.,
A support element, e.g. 203, etc., integral with the housing 102, etc. or mechanically connected to the housing 102, etc. and
A container retaining space for receiving a container comprising a drug Dr,
wherein the support element 203, etc. may be configured to support the container within the housing 102, etc.,
wherein the drug delivery device 100 to 1100 may comprise at least one spacer element 214, 450, 650, 794, 795, etc. or may be adapted to interact with at least one spacer element 450, 650, 794, etc.,
wherein the drug delivery device 100 to 1100 may be configured such that in a first state of the drug delivery device 100 to 1100, a first axial position UP of the container relative to the housing 102, etc. is adjusted by the at least one spacer element 214, 450, 650, 794, 795, etc. being in a first spacer position,
and that in a second state of the drug delivery device 100 to 1100, a second axial position LP of the container relative to the housing 102, etc. may be adjusted by the at least one spacer element 214, 450, 650, 794, 795, etc. being in a second position within the housing 102, etc. or being outside of the housing 102, etc.,
wherein the first axial position UP may enable a smaller injection depth, e.g. D3B3, of a needle 110, etc. coupled to the container compared to the injection depth, e.g. D2B3, enabled if the container is in the second axial position LP.

The spacer element 450, 650 may be a bifurcated spacer element 450, 650 comprising a basis portion 452, 652, a first pronged portion 454 and a second pronged portion 456 extending in parallel from the basis portion 452, 652 and forming an intermediate space 458 between the first pronged portion 454 and the second pronged portion 456. The lateral width Wi2 of the intermediate space 458 may greater than the lateral width of the diameter D2 of a neck portion 436, 636 of the container at a position close to a larger diameter portion of a barrel 432 of the container.

The at least one spacer element may comprise:
a) A first class lever 794, 795 comprising;
   An elongated operating portion 794a, 795a,
   An elongated spacer portion 794c, 795c configured to interact with at least one of the container or a carrier of the container, and
   A mounting portion 794b, 795b arranged between the operating portion 794a, 795a and the spacer portion 794c, 795c and comprising a pivotable mounting element,
b) A second class lever 894, 895 comprising:
   An elongated operating portion 894a, 895a,
   An elongated spacer portion 894b, 895b configured to interact with at least one of the container or a carrier of the container, and
   A mounting portion 894c, 895c comprising a pivotable mounting element, wherein the elongated spacer portion 894b, 895b is arranged between the elongated operating portion 894a, 895a and the mounting portion 894c, 895c,
c) At least one cam element 994, 995, 1099a comprising:
   A mounting portion 994c, 995c comprising a pivotable mounting element,
   A curved outer surface 994a, 995a adapted to interact with an operating element 991a, 993a, and
   An inner spacer portion 994b, 995b configured to interact with at least one of the container or a carrier of the container.

In some embodiments, no further resilient element(s) except e.g. the ones already mentioned may be necessary to move levers and/or cams etc. forth and back. In certain embodiments, additional resilient elements can be used, adapted, and configured for this purpose, e.g. to move levers and/or cams etc. forth and back.

The drug delivery device 100 to 1100 may comprise an axially movable needle protection element 408, 508x, 608, etc. and
a) at least one resilient element 460, 560x, 660, preferably only one resilient element 460, 560x, 660, configured to bias the axially movable needle protection element 408, 508x, 608 in the distal D direction,
wherein the drug delivery device 400, 500x, 600 may be configured such that a proximal end of the at least one resilient element 460, 560x, 660 is arranged on an abutting face 465, 565, 665 that is located distally compared to the at least one spacer element 450, 650, 794c etc. or to a retaining space for the at least one spacer element 450, 650, and/or
b) wherein the drug delivery device 500 or 100 to 1100 may comprise at least two resilient elements 561, 562 configured to bias the axially movable needle protection element 508, etc. in the distal D direction,
wherein the proximal ends of the at least two resilient elements 561, 562 may be arranged on at least one abutting face 566, 567 that may be located proximal compared to the at least one spacer element 450, 650 or to a retaining space for the at least one spacer element 450, 650, 794c, etc. and
wherein the at least two resilient elements 561, 562 may be arranged laterally with regard to the at least one spacer element or to a retaining space for the at least one spacer element 450, 650, 794c, etc. and/or to an operating element of the at least one spacer element.

Adjustment of injection depth of an autoinjector achieved through spacer attached to contact point with syringe shoulder is provided.

As is illustrated e.g. in FIGS. 2A to 3B, an autoinjector design may be adapted by:
A spacer or mechanism which achieves a shift in injection depth range through contact with the ‘shoulder’ of the pre-filled syringe,
A body designed to locate and support the syringe and said mechanism, and
Additional parts designed to allow the user to easily select an injection depth setting.

Variations of this design may form variations of the embodiments including:
1. A spacer located in between the body which is installable/removable by the user, and
2. A mechanism located within the body which changes location of the syringe though a change in thickness and/or profile achieved by external adjustment by the user via:
   1. Levers located in the body which change in profile, and
   2. Other possible variations thereof.

A mechanism may be used that is located within the body which changes location of the syringe by a change in thickness and/or profile, e.g. height, achieved by external adjustment by the user via levers located in the body which change contact surface when moved.

In this embodiment of a previous design mentioned above an external input may cause one or more (two shown) levers to move, changing the contact area with the syringe shoulder.

Exemplary, FIGS. 7A to 9C illustrate a possible implementation whereby a rotational input from an external ring, e.g. 790, causes rotation of a lever element, the surface of which is in contact with the syringe shoulder initially at a lower position, and once rotated, moves the syringe towards the proximal end of the device. The reverse may also be considered, e.g. whereby the syringe is moved distally by a counter rotation of e.g. the external ring.

Variations of mechanical means to change syringe contact point may comprise:
The embodiment detailed previously whereby a first class lever is used to change contact position, see FIGS. 8C and 9C.
A similar embodiment according to FIGS. 10A and 10B whereby a second class lever is used to change contact position.
An arrangement according to FIGS. 11A to 11C comprises a feature on the ring that interacts with a cam profile in order to change the contact position. It is considered that this implementation could include multiple interlinked cams in a similar configuration to a shutter mechanism to increase contact area.

Details of possible external appearance of ring feature are illustrated e.g. in FIG. 12. It is considered that the ring, e.g. 1190, may have physical and visual feedback for various depth adjustment levels.

Modification of the plunger rod length by the spacer height may not be necessary. Accidental expelling of drug Dr may be more of a concern in design where the user is able to move the syringe toward the proximal end of the device, e.g. in the steps identified in FIG. 9B, although clearance can be made through the design and/or selection of an appropriate plunger length. Plungers of different lengths may be explored.

The drug delivery device may have a nominal gap from plunger to stopper of 5 mm, and a minimum stopper gap of around 1 mm due to potential tolerances in the device. These tolerances are mainly from variations in the fill level of the syringe. It may be possible to increase the stopper gap in order to accommodate for subsequent potential change in the position of the syringe. This may come at the cost of increased impact force which can be a subject of further review at a later stage of development and is related to other factors e.g. drug Dr viscosity and strength of the drive spring. Impact force could also be mitigated by other means.

Although embodiments of the present disclosure and their advantages have been described in detail, it should be understood that various changes, substitutions and alterations can be made therein without departing from the spirit and scope of the disclosure as defined by the appended claims. For example, it will be readily understood by those skilled in the art that many of the features, functions, processes and methods described herein may be varied while remaining within the scope of the present disclosure. Moreover, the scope of the present application is not intended to be limited to the particular embodiments of the system, process, manufacture, method or steps described in the present disclosure. As one of ordinary skill in the art will readily appreciate from the disclosure of the present disclosure, systems, processes, manufacture, methods or steps presently existing or to be developed later that perform substantially the same function or achieve substantially the same result as the corresponding embodiments described herein may be utilized according to the present disclosure. Accordingly, the appended claims are intended to include within their scope such systems, processes, methods or steps. Further, it is possible to combine embodiments mentioned in the first part of the description with examples of the second part of the description which relates to FIGS. 1 to 12.

### REFERENCE NUMERALS

A longitudinal axis
100 drug delivery device
101 container retaining member
102 main housing part
104 piston rod
106 driving mechanism
108 actuating element
110 needle
112 cap
D distal end
P proximal end
Dr drug
SU1, SU2 spacer unit
200 drug delivery device
202 main housing part
202a protrusion
202b distal portion
202c proximal portion
203 central part of main housing part
204 piston rod
204a first protrusion
204b second protrusion
206 driving mechanism
208 needle shroud
208a distal portion
208b arms
208c proximal portion
208d proximal aperture
208e protrusion
208f distal aperture (cooperates with protrusion 202a)
210 needle
212 resilient syringe holder
214 spacer unit
216 optional operating element of distance unit
218 distance portion
220 skin
222 upper layer
224 target layer
230 syringe
232 barrel portion
234 shoulder
236 distal portion
D2A1 first remaining distance
D2A2 first axial distance
D2A3 insertion depth
D2B1 second remaining distance (zero mm)
D2B3 insertion depth during injection, injection depth
300 axial shift of syringe 230
301 axial shift of needle 210
D3A1 first remaining distance
D3A2 second axial distance
D3A3 insertion depth
D3B1 second remaining distance
D3B2 insertion depth during injection
400 drug delivery device
402 main housing part
403 central part of main housing part
403a bottom of central part 403
408 needle shroud
408a distal portion
408b1, 408b2 arms
430 syringe
432 barrel
434 shoulder
436 distal portion
440 to 443 axial rib
A1, A2 first, second transversal axis
444 to 446 aperture
444 to 446 apertures
448 wall portion
450 bifurcated spacer
452 basis portion
454 first prong
45€ second prong
458 opening
460 resilient element
465 abutting face
R ramp
470 insertion direction
Wi1 lateral width of bifurcated spacer
Wi2 lateral width of aperture for bifurcated spacer
T1 thickness
D1 outer diameter of barrel
D2 outer diameter of neck portion
D4B, D4D distance
HL horizontal line
500, 500x drug delivery device
502, 502x main housing part
503, 503x central part
508, 508x actuating element, e.g. needle shroud
508a, 508ax distal portion
510, 510x needle
520, 520x surface of skin
530, 530x syringe
532, 532x barrel
534, 534x shoulder
536, 536x distal portion
536, 536x distal portion
544 to 546 aperture
544x to 546x aperture
560, 560x compression spring
561, 562 first compression spring, second compression spring
565 abutting face
566, 566a abutting face
567, 567a abutting face
566xa, 567xa abutting face
580 syringe carrier
582, 584 distal arms
D5A, D5B distance
600 drug delivery device
601 drug window
602 main housing part
603 central part/portion
603a bottom
608 actuating element, e.g. needle shroud
610 needle
630 syringe
632 barrel
634 shoulder
636 distal portion of syringe
644 to 646 aperture
650 bifurcated spacer element
651a first thickness portion
651b second thickness portion
659a first identifier
659b second identifier
660 compression spring
665 abutting face
R1a, R1b, R2a, R2b ramp
T1b, T2 thickness
700 drug delivery device
702 main housing part
703 central part
703a, 703b radial protrusions on central part
703c, 703d aperture
703e, 703f stop face
703g, 703h slanted face
730 syringe
732 barrel portion
734 shoulder
736 distal portion
740 to 744 axial/radial rib
790 ring
791 first ring/lever interface
791a, 791b protrusion
792 second ring/lever interface
792a, 792b protrusion
794 first lever
795 second lever
794a operating portion
794b hinge
794c spacer arm
795a operating portion
795b hinge
795c spacer arm
R3 ramp
W1 angle
LP lower position
UP upper position
796 operation protrusion
D8B distance
D8C minimum radial distance
D9B distance
D9C minimum radial distance (second state)
W2 angle
Arr2 to Arr12 arrow
AP1 first angular position
AP2 second angular position
800 drug delivery device
802 main housing part
803 central part
808b1, 808b2 arm of needle shroud
836 distal portion of syringe
840 axial/radial rib
890 ring
891 first ring/lever interface
892 second ring/lever interface
894 first lever
894a operating end
894b spacer arm
894c hinge
895 first lever
895a operating end
895b spacer arm
895c hinge
D10A minimum radial distance (first state)
D10B minimum radial distance (second state)
W3 angle
Arr13, Arr14 arrow
AP3 third angular position
AP4 fourth angular position
900 drug delivery device
902 main housing part
903 central part
908b1, 908b2 arm of needle shroud
936 distal portion of syringe
940 axial/radial rib
990 ring
991 first cam/protrusion interface
991a protrusion
992 second cam/protrusion interface
992a protrusion
994 first cam element
994c hinge
995 second cam element
995c hinge
Arr15, Arr16 arrow
W4 angle
AP5 fifth angular position
AP6 sixth angular position
D11A minimum radial distance (first state)
D11B minimum radial distance (first state)
1000 drug delivery device
1002 main housing part
1003 central part
1008b1, 1008b2 arm of needle shroud
1036 distal portion of syringe
1040 axial/radial rib
1090 ring
1091 first interface
1091a protrusion
1092 second interface
1092a protrusion
1094 first spacer element
1095 second spacer element
1099a to 1099f further spacer element(s)
1100 drug delivery device
1100a hand
1100b thumb
1100c index finger
1100d middle finger
1100e ring finger
1101 drug window
1102 main housing part
1108 needle shroud (actuating element)
1190 ring
1190a marker
1190b identifiers

1-15. (canceled)

16. A drug delivery device with adjustable injection depth, the drug delivery device comprising:
a housing,
a support element integral with the housing or mechanically connected to the housing, and
a container retaining space for receiving a container comprising a drug,
wherein the support element is configured to support the container within the housing,
wherein the drug delivery device comprises at least one spacer element or is adapted to interact with at least one spacer element,
wherein the drug delivery device is configured such that in a first state of the drug delivery device, a first axial position of the container relative to the housing is adjusted by the at least one spacer element being in a first spacer position, and that in a second state of the drug delivery device, a second axial position of the container relative to the housing is adjusted by the at least one spacer element being in a second position within the housing or being outside of the housing,
wherein the first axial position enables a smaller injection depth of a needle coupled to the container compared to the injection depth enabled if the container is in the second axial position,
wherein the at least one spacer element is configured to be translationally movable crosswise to a longitudinal axis of the drug delivery device from the first spacer position to the second spacer position or to a position outside of the drug delivery device.

17. The drug delivery device according to claim 16, wherein the at least one spacer element is configured to be completely removable from the drug delivery device by a user.

18. The drug delivery device according to claim 16, wherein the at least one spacer element is built into the housing, and
wherein the at least one spacer element is configured to be movable from the first spacer position to the second spacer position using at least one operating element.

19. The drug delivery device according to claim 16, wherein the spacer element is a bifurcated spacer element comprising a basis portion, a first pronged portion, and a second pronged portion extending in parallel with the first pronged portion from the basis portion and forming an intermediate space between the first pronged portion and the second pronged portion.

20. The drug delivery device according to claim 19, wherein the lateral width of the intermediate space is greater than the lateral width or the diameter of a neck portion of the container.

21. The drug delivery device according to claim 20, wherein the spacer element comprises at least one ramped portion on at least one of the following: on a free end of the first pronged portion or on a free end of the second pronged portion or on at least one intermediate portion of the first pronged portion or on at least one intermediate portion of the second pronged portion, and
wherein the spacer element comprises at least one portion of constant thickness on at least one of the following: on the basis portion or on the first pronged portion or on the second pronged portion.

22. The drug delivery device according to claim 16, comprising:
an axially movable needle protection element, and
at least one resilient element configured to bias the axially movable needle protection element in a distal direction,
wherein the drug delivery device is configured such that a proximal end of the at least one resilient element is arranged on an abutting face that is located distally compared to the at least one spacer element or to a retaining space for the at least one spacer element.

23. The drug delivery device of claim 16, comprising:
an axially movable needle protection element, and
at least two resilient elements configured to bias the axially movable needle protection element in a distal direction,
wherein proximal ends of the at least two resilient elements are arranged on at least one abutting face that is located proximal compared to the at least one spacer element or to a retaining space for the at least one spacer element, and
wherein the at least two resilient elements are arranged laterally with regard to the at least one spacer element or to a retaining space for the at least one spacer element and/or to an operating element of the at least one spacer element.

24. A drug delivery device with adjustable injection depth, the drug delivery device comprising:
a housing,
a support element integral with the housing or mechanically connected to the housing, and
a container retaining space for receiving a container comprising a drug,
wherein the support element is configured to support the container within the housing,
wherein the drug delivery device comprises at least one spacer element or is adapted to interact with at least one spacer element,
wherein the drug delivery device is configured such that in a first state of the drug delivery device, a first axial position of the container relative to the housing is adjusted by the at least one spacer element being in a first spacer position, and that in a second state of the drug delivery device, a second axial position of the container relative to the housing is adjusted by the at least one spacer element being in a second position within the housing,
wherein the first axial position enables a smaller injection depth of a needle coupled to the container compared to the injection depth enabled if the container is in the second axial position,
wherein the at least one spacer element is configured to be pivotable from the first position to the second position.

25. The drug delivery device according to claim 24, wherein:
the drug delivery device comprises a rotatable operating feature configured to be rotated by a user of the drug delivery device, and
the rotatable operating feature is configured to interact with the pivotable spacer element.

26. The drug delivery device according to claim 24, wherein the at least one spacer element comprises:
a) a first class lever comprising:
an elongated operating portion,
an elongated spacer portion configured to interact with at least one of the container or a carrier of the container, and
a mounting portion arranged between the operating portion and the spacer portion and comprising a pivotable mounting element,
b) a second class lever comprising:
an elongated operating portion,
an elongated spacer portion configured to interact with at least one of the container or a carrier of the container, and
a mounting portion comprising a pivotable mounting element,
wherein the elongated spacer portion is arranged between the elongated operating portion and the mounting portion, and
c) at least one cam element comprising:
a mounting portion comprising a pivotable mounting element,
a curved outer surface adapted to interact with an operating element (991a, 993a), and
an inner spacer portion configured to interact with at least one of the container or a carrier of the container.

27. The drug delivery device according to claim 24, wherein the at least one spacer element comprises a curved portion, and
wherein the at least one spacer element comprises at least one ramp feature configured to move or to allow to move the container or the container and a carrier of the container from the first axial position to the second axial position.

28. The drug delivery device according to claim 24, wherein the at least one spacer element comprises a proximal facing first face configured to abut the container and a distally facing second face configured to abut the housing or the support element or another element of the drug delivery device.

29. The drug delivery device according to claim 28, wherein:
the container comprises a barrel portion comprising a first diameter, a distal neck portion and a shoulder portion arranged between the barrel portion and the distal neck portion,
the neck portion comprises a second diameter that is smaller than the first diameter, and
the first surface is configured to abut the shoulder portion.

30. The drug delivery device according to claim 28, wherein the container comprises a barrel portion comprising a first diameter and a flange portion comprising a maximum second diameter that is greater than the first diameter, and the first surface is configured to abut the flange portion.

31. The drug delivery device according to claim 28, further comprising a container carrier comprising a main carrier portion encompassing the retaining space or the container, the container carrier comprising a proximal flange portion and at least one distal arm extending distally from the main carrier portion.

32. The drug delivery device according to claim 31, wherein the first face is configured to abut the distal end of the container carrier, where the distal end of the container carrier comprises a distal end of at least one of the at least one distal arms.

33. The drug delivery device according to claim 31, wherein the second face is configured to abut to a proximal part of the container carrier, wherein the proximal part of the container carrier comprises a proximal part of the flange portion of the container carrier.

34. The drug delivery device according to claim 24, wherein the container comprises a drug.

35. A method of injecting a drug from a drug delivery device, the method comprising:
providing a drug delivery device with adjustable injection depth, the drug delivery device comprising:
a housing,
a support element integral with the housing or mechanically connected to the housing, and
a container retaining space for receiving a container comprising a drug,
wherein the support element is configured to support the container within the housing,
wherein the drug delivery device comprises at least one spacer element or is adapted to interact with at least one spacer element,
wherein the drug delivery device is configured such that:
in a first state of the drug delivery device a first axial position of the container relative to the housing is adjusted by the at least one spacer element being in a first spacer position, and
in a second state of the drug delivery device, a second axial position of the container relative to the housing is adjusted by the at least one spacer element being in a second position within the housing or being outside of the housing,
wherein the first axial position enables a smaller injection depth of a needle coupled to the container compared to the injection depth enabled if the container is in the second axial position,
a) moving the at least one spacer element translational crosswise to a longitudinal axis of the drug delivery device from the first spacer position to the second spacer position or to a position outside of the drug delivery device, or
b) pivoting the at least one spacer element from the first position to the second position, and
injecting the drug.
